(19) United States
(12) Patent Application Publication
(10) Pub. No.: US 2026/0348801 A1
(43) Pub. Date: Nov. 5, 2026

(54) TYRAMIDE AND TYRAMIDE ANALOG CONJUGATES AND USES THEREOF FOR BIOLOGICAL TARGET STAINING PROCEDURES

(71) Applicant: LIFE TECHNOLOGIES CORPORATION, Carlsbad, CA (US)

(72) Inventors: Chris VONNEGUT, Springfield, OR (US); Scott CLARKE, Eugene, OR (US); Shih-Jung HUANG, Eugene, OR (US); Nancie MOONEY, Eugene, OR (US)

(21) Appl. No.: 19/114,885

(22) PCT Filed: Dec. 18, 2023
(86) PCT No.: PCT/US2023/084559

§ 371 Date: Mar. 25, 2025

Related U.S. Application Data
(60) Provisional application No. 63/477,653, filed on Dec. 29, 2022.
(60) Provisional application No. 63/485,651, filed on Feb. 17, 2023.

(51) Int. Cl. G01N 1/30; C07C 235/66; C07C 309/60

(52) U.S. Cl. CPC G01N 1/30; C07C 235/66; C07C 309/60; G01N 2001/302

(57) ABSTRACT

The present invention provides compounds and compositions suitable for target visualization, such as immunostaining procedures, that form covalent colorimetric and/or fluorescent stains. Certain disclosed compounds have Formula I, (P¹)ₙ-L-P², where P¹ is a first aromatic ring system comprising at least two fused rings, wherein at least one of the fused rings comprises a structure represented as, P² is a second aromatic ring system substituted with at least one —OH group, L is a linker and n=1-20. Particular examples of a compound satisfying both Formula I and Formula II include Compounds according to the present invention are useful for forming covalent conjugates at or about a biological target. Embodiments of target staining protocols comprise providing at least one disclosed compound, and using the compound to detect a target in a sample. Kits useful for detecting biological targets also are disclosed.

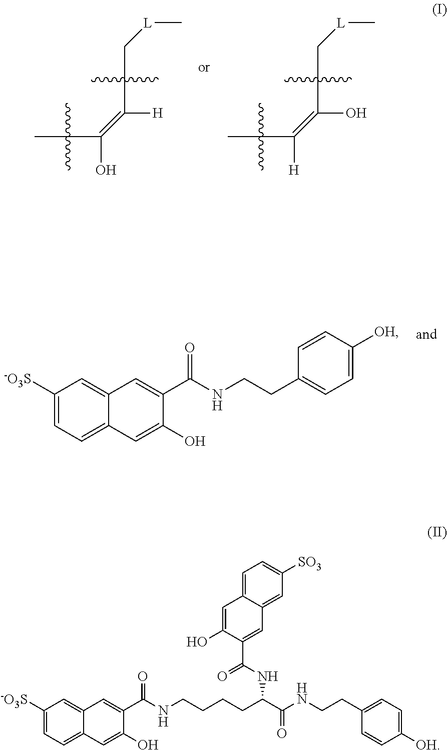

### RELATED APPLICATIONS

The present application includes subject matter that is related to subject matter disclosed by U.S. provisional application No. 63/477,653, filed Dec. 29, 2022, and U.S. provisional application No. 63/485,651, filed Feb. 17, 2023, which are incorporated herein by reference.

### FIELD

The present disclosure concerns compounds, compositions comprising such compounds, and embodiments of a method for using such compounds and compositions for staining procedures for visualizing biological targets, and more particularly concerns fused-ring tyramide conjugates, such as naphthol-tyramide conjugates, suitable for forming covalent colorimetric stains for in situ hybridization (ISH) and immunostaining procedures.

### BACKGROUND

Methods for detecting a target, such as a protein target in a biological sample, are commonly used for research purposes and to analyze pathology samples, such as formalin-fixed and paraffin-embedded samples that are used for diagnosis and for tracking disease progress. Specific binding agents, such as antibodies, can be produced that bind specifically to a desired antigen. This allows detecting small amounts of an antigen within a sample. It then becomes necessary to visualize the antibody-antigen complex. This can be achieved in a number of ways, such as by using a chromophore that is deposited at a target. Procedures for visualizing biological targets may be facilitated by using permanent, covalent markers, particularly those that produce distinctive colors. For example, for IHC protocols that involve hematoxylin, the counterstain can be hard to distinguish from DAB (3,3′-diaminobenzidine) stains. Furthermore, other reagents, such as AEC (3-amino-9-ethylcarbozole), may be soluble in solvents commonly used to rinse samples, and accordingly stains produced by AEC IHC protocols wash out.

As a result, a need exists for compounds and compositions suitable for staining procedures that form covalent colorimetric stains. These compounds and compositions preferably would provide a number of advantages, such as: (1) similar or sharper staining patterns and intensity as DAB or Fast Red; (2) similar permanent staining as that provided by DAB that are resistant to solvent washes, and mountable in aqueous or organic mountants; (3) covalently attaching on or around the target, as opposed to simply precipitating; (4) producing permanent stains in distinctive colors, such as red, purple, blue, and green; and (5) allowing fluorescence detection in addition to colorimetric detection.

### SUMMARY

The present invention provides compounds and compositions suitable for target visualization, such as immunohistochemistry (IHC) staining procedures that form covalent colorimetric stains. Certain disclosed embodiments concern using compounds satisfying a Formula I

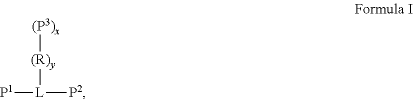

where L is a linker having from 1 to at least 50 atoms, more typically 2 to 20 atoms, in a chain; P¹, P² and P³ are aromatic rings or aromatic ring systems; R is a bond, or is independently an L group; and x and y independently are 0 to 5. The P¹ and P³ ring systems may be the same or different and each may comprise at least two fused rings. Typically, at least one of the P¹ and/or P³ fused ring systems comprises a structure represented as

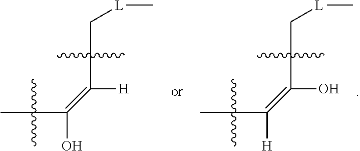

For certain embodiments, P² is a phenyl ring substituted with at least one —OH group. Compounds suitable for use with the present invention also may satisfy a Formula II

P¹-L-P² Formula II,

where L, P¹ and P² are as stated for Formula I.

More particularly, P¹ and P³ typically are hydrocarbon and/or heteroaryl ring systems. Typical hydrocarbon ring systems have from 2 to 4 rings in a fused-ring system. Suitable exemplary hydrocarbon ring systems include naphthol, anthracenol, tetracenol, and phenanthrol. Heteroaryl fused ring systems preferably are electron rich, with examples including fused furan, fused thiophene, and fused pyrrole ring systems. P¹ and/or P³ may be substituted with at least one moiety selected to increase solubility in solvents commonly used for methodologies such as IHC, such as an aqueous solubility moiety selected to increase the aqueous solubility of the compound. Exemplary solubility moieties include SO₃⁻, —SO₃H, —CO₂⁻, —CO₂H, —HPO₃⁻², H₂PO₃⁻¹, H₃PO₃, PEG,

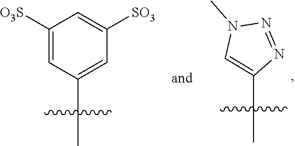

or salt forms thereof. L is selected from a covalent bond. C₁-C₅₀ aliphatic, C₁-C₅₀ alkyl, a C₁-C₅₀ alkyl amide, a C₁-C₅₀ ketone, a C₁-C₅₀ glycol or polyol. The C₁-C₅₀ chain length is more typically C₁-C₂₀, and even more particularly C₃-C₁₅. One exemplary linker family has a formula

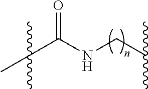

where n is from 1 to 10, more typically 2-5, with one particular linker having L as

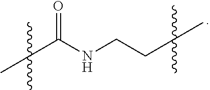 [SMILES: *CCNC(*)=O]

Compounds according to the present invention may also satisfy Formula III

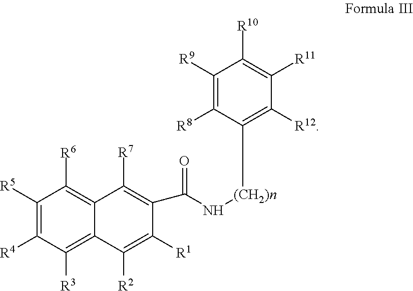

With reference to Formula III, R¹ and R² are independently selected from H and OH; R³-R⁸ are independently selected from H, —F, —Cl, —Br, —I, —OH, —OR⁹ where R⁹ is C₁-C₆ alkyl, —SO₃⁻, —SO₃H, —CO₂⁻, —CO₂H, —HPO₃⁻², H₂PO₃⁻¹, H₃PO₃, PEG,

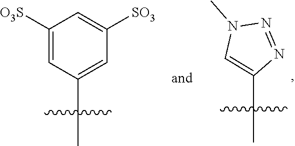

or salt forms thereof, where at least one of R¹, R², R⁷ and at least one of R⁸-R¹² is hydroxyl. Alternatively, R³ and R taken together form at least one fused ring system; and/or R⁴ and R⁵ taken together form at least one fused ring system; and/or R⁵ and R⁶ taken together form at least one fused ring system; and n is 1 to 5.

Particular examples of compounds satisfying Formulas I, II and/or III include

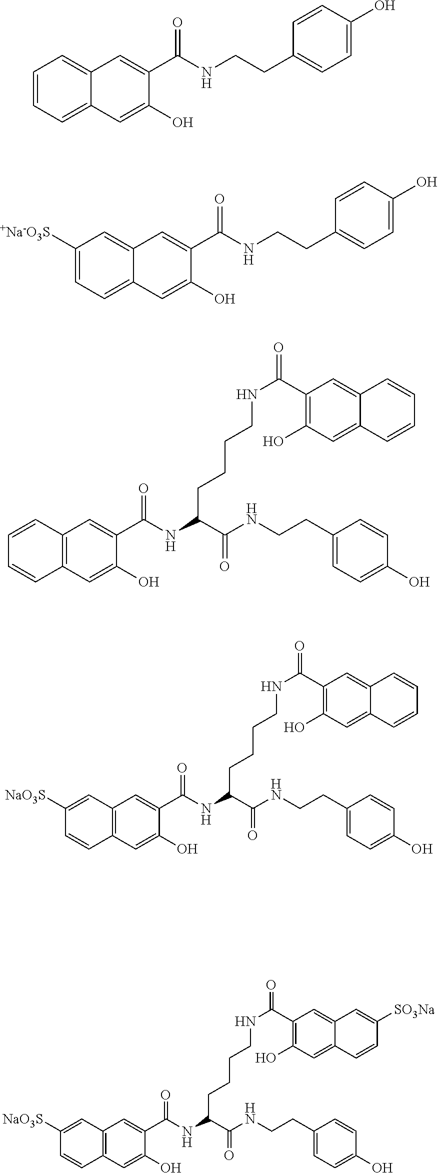

Compounds according to the present invention are useful for forming covalent conjugates at or about a biological target. The biological target may be a cell, a tissue, a virus, a gene, an epitope, a target genetic sequence, or a target protein sequence. Accordingly, the present invention also concerns covalent conjugates that comprise a biological sample comprising a target and at least one compound according to Formula I, Formula II and/or Formula III covalently associated with the target.

Embodiments of a method for using disclosed compounds in target staining protocols also are disclosed. One embodiment of the method comprises providing at least one compound according to Formula I, Formula II and/or Formula III; and using the compound to detect a target in a sample. For example, the method may comprise contacting a biological sample to covalently associate a first compound to or substantially adjacent the target. The first compound is then contacted with a diazonium compound under conditions that covalently link the first compound to the diazonium compound to form conjugate and allow detection of the target. The first compound may exhibit a first optical signal upon irradiation at an appropriate wavelength of light, and the conjugate may exhibit a second optical signal upon irradiation at an appropriate wavelength of light, wherein the first optical signal and the second optical signal are different.

The method may involve contacting the target with the first compound in the presence of a peroxidase to covalently couple the first compound to the target. Suitable peroxidases include, without limitation, horseradish peroxidase, microperoxidases, or ascorbate peroxidase (APEX). Exemplary diazonium compounds include Fast Red, Fast Violet, and Fast Blue.

The method may be an IHC procedure. For example, the biological target may be recognized by a primary antibody. For these embodiments, the primary antibody may be coupled to a peroxidase. The method comprises contacting the biological target with a primary antibody that associates with the target; and contacting the primary antibody with a secondary antibody to the primary antibody to form a primary-secondary antibody conjugate, wherein the secondary antibody is bound to a peroxidase. Exemplary primary antibodies include an ESR1 antibody, a KRT15 antibody, and/or a CDH17 antibody. For certain embodiments, the primary antibody was a mouse antibody, and the secondary antibody was a poly HRP-goat anti-mouse (GAM); or the primary antibody was a rabbit antibody, and the secondary antibody was a poly-HRP-goat anti-rabbit (GAR). The target may be detected colorimetrically, such as by brightfield microscopy, fluorescence, or both.

The present invention also provides kits useful for detecting biological targets. One example of a kit according to the present invention comprises a compound according to Formula I, Formula II and/or Formula III, and a diazonium salt. A more particular example of a kit according to the present invention comprises 3% Bovine Serum Albumin; poly HRP-goat anti-mouse (GAM), poly-HRP-goat anti-rabbit (GAR), and/or HRP-conjugated streptavidin; a compound according to Formula I, II and/or Formula III, such as a 5 mM aqueous solution thereof; a buffer system for tyramide deposition, such as a 200 mM citrate buffer, pH 4, and 7.5% stabilized hydrogen peroxide; a diazonium salt, such as Fast Red TR or Fast Blue BB; and a color development buffer, such as 100 mM Tris, pH 8.5.

The foregoing and other objects, features, and advantages of the invention will become more apparent from the following detailed description, which proceeds with reference to the accompanying figures.

### BRIEF DESCRIPTION OF THE DRAWINGS

FIG. 1 is a flow chart for one embodiment of a method according to the present invention.

FIG. 2 is a flow chart illustrating the steps of FIG. 1, but with reference to using a primary antibody to recognize the target, a secondary antibody associated with horseradish peroxidase (HRP) that recognizes the primary antibody, covalent deposition of a tyramide compound according to the present invention, and then imaging using a diazonium salt that reacts with the tyramide compound.

FIG. 3 is a schematic drawing illustrating one embodiment of a method according to the present invention for associating a tyramide compound according to the present invention with a biological sample in the presence of a horse radish peroxidase and H₂O₂.

FIG. 4 is a brightfield image of Peyer's Patches (groupings of lymphoid follicles in the mucus membrane that line the small intestine) stained with a tyramide compound according to the present invention.

FIG. 5 is a brightfield image of Peyer's Patches stained with DAB for comparison to FIG. 3.

FIG. 6 is a brightfield image of intestinal villi stained with a tyramide compound according to the present invention.

FIG. 7 is a brightfield image of intestinal villi stained with DAB for comparison to FIG. 5.

FIG. 8 is a fluorescence image of intestinal villi stained with a tyramide compound according to the present invention.

FIG. 9 is a DAPI counterstained image of intestinal villi.

FIG. 10 is a brightfield image of intestinal villi with a hematoxylin counterstain.

FIG. 11 provides staining images that illustrate results obtained using a Formula I

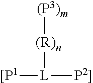

compound according to the present invention at 0.5 mM and 1 mM concentrations and 10-minute and 30-minute incubation times.

FIG. 12 provides staining images comparing the results obtained using: a first formula II [P¹-L-P²] compound according to the present invention, namely

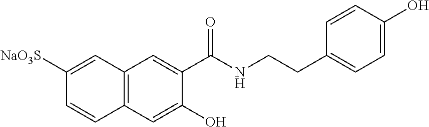

in a 1 mM staining solution that provided a total compound concentration of 1 mM; a second Formula I

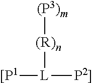

compound according to the present invention, namely

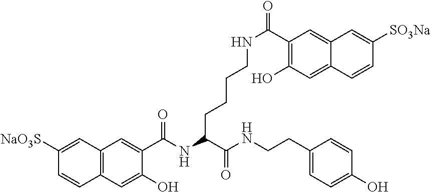

in an 0.5 mM staining solution that provided a total compound concentration of 1 mM; and the well-known diaminobenzidine (DAB).

FIG. 13 provides tissue staining images (20×) obtained using disclosed embodiments of the present invention illustrating that such embodiments are compatible for use with formalin-fixed, paraffin-embedded (FFPE) tissue and organic mountants.

FIG. 14 provides tissue staining images (20×) of mounted cryo tissue obtained using disclosed embodiments of the present invention illustrating that such embodiments are compatible for use with cryo-preserved tissue.

FIG. 15 provides tissue staining images (20×) of FFPE tissue obtained using disclosed embodiments of the present invention illustrating that such embodiments are compatible for use with aqueous mountants.

FIG. 16 provides tissue staining images (20×) of FFPE breast tissue (ESRI) obtained using disclosed embodiments of the present invention illustrating fluorescence performance, with fluorescence at greater than 3 times the background intensity.

FIG. 17 provides tissue staining images (20×) of FFPE tissue obtained using disclosed embodiments of the present invention illustrating that such embodiments are compatible with hematoxylin.

FIG. 18 provides tissue staining images (20×) of FFPE tissue obtained using disclosed embodiments of the present invention illustrating that such embodiments are compatible with eosin.

FIG. 19 provides tissue staining images (20×) of FFPE tissue obtained using disclosed embodiments of the present invention illustrating that such embodiments are compatible with Methyl Green.

FIG. 20 provides cryo tonsil tissue staining images (20×) mounted with Cytoseal 60 obtained using disclosed embodiments of the present invention illustrating dual staining with DAB.

FIG. 21 provides cryo mouse intestine tissue staining images (20×) mounted with Cytoseal 60 obtained using disclosed embodiments of the present invention illustrating dual staining with DAB.

FIG. 22 provides cryo mouse intestine tissue staining images (20×) mounted with Cytoseal 60 obtained using disclosed embodiments of the present invention illustrating dual staining capability.

FIG. 23 provides cryo mouse intestine tissue staining images (20×) mounted with Cytoseal 60 obtained using disclosed embodiments of the present invention illustrating dual staining-fluorescence capability.

FIG. 24 provides brain NeuN tissue staining images (20×) obtained using disclosed embodiments of the present invention illustrating blue IHC staining capability for an aqueous mount.

FIG. 25 provides brain NeuN tissue staining images (20×) obtained using disclosed embodiments of the present invention illustrating blue IHC staining capability for an organic mount.

FIG. 26 provides brain NeuN tissue staining images (20×) obtained using disclosed embodiments of the present invention illustrating red IHC staining capability for an aqueous mount.

FIG. 27 provides brain NeuN tissue staining images (20×) obtained using disclosed embodiments of the present invention illustrating red IHC staining capability for an organic mount.

FIG. 28 compares cryo breast tissue, ESRI, red staining images (20×) obtained using disclosed embodiments of the present invention and DAB relative to a no primary control.

FIG. 29 compares cryo breast tissue, ESRI, blue staining images (20×) obtained using disclosed embodiments of the present invention and DAB relative to a no primary control.

FIG. 30 compares cryo tonsil tissue. KRT15, red staining images (20×) obtained using disclosed embodiments of the present invention and DAB relative to a no primary control.

FIG. 31 compares cryo tonsil tissue, KRT15, blue staining images (20×) obtained using disclosed embodiments of the present invention and DAB relative to a no primary control.

FIG. 32 compares cryo colon tissue, CDH17, red staining images (20×) obtained using disclosed embodiments of the present invention and DAB relative to a no primary control.

FIG. 33 compares cryo colon tissue, CDH17, blue staining images (20×) obtained using disclosed embodiments of the present invention and DAB relative to a no primary control.

FIG. 34 provides no antibody control red and blue staining images (20×) for cryo colon tissue, CDH17, obtained using disclosed embodiments of the present invention and DAB.

FIG. 35 compares FFPE breast, ESR1, blue staining images (20×) obtained using disclosed embodiments of the present invention and DAB relative to a no primary control.

FIG. 36 compares FFPE tonsil, KRT15, red staining images (20×) obtained using disclosed embodiments of the present invention and DAB relative to a no primary control.

FIG. 37 compares FFPE tonsil, KRT15, blue staining images (20×) obtained using disclosed embodiments of the present invention and DAB relative to a no primary control.

FIG. 38 compares FFPE colon, CDH17, red staining images (20×) obtained using disclosed embodiments of the present invention and DAB relative to a no primary control.

FIG. 39 compares FFPE colon, CDH17, blue staining images (20×) obtained using disclosed embodiments of the present invention and DAB relative to a no primary control.

FIG. 40 compares FFPE breast, ESR1, red staining images (20×), aqueous mountant, no dehydration/clearing, obtained using disclosed embodiments of the present invention and DAB relative to a no primary control.

FIG. 41 compares FFPE breast, ESR1, red staining images (20×), aqueous mountant, no dehydration/clearing, obtained using disclosed embodiments of the present invention and DAB relative to a no primary control.

FIG. 42 compares FFPE breast, KRT15, red staining images (20×), aqueous mountant, no dehydration/clearing, obtained using disclosed embodiments of the present invention and DAB relative to a no primary control.

FIG. 43 compares FFPE tonsil, KRT15, blue staining images (20×), aqueous mountant, no dehydration/clearing, obtained using disclosed embodiments of the present invention and DAB relative to a no primary control.

FIG. 44 compares FFPE colon, CDH17, red staining images (20×), aqueous mountant, no dehydration/clearing, obtained using disclosed embodiments of the present invention and DAB relative to a no primary control.

FIG. 45 compares FFPE colon, CDH17, blue staining images (20×), aqueous mountant, no dehydration/clearing, obtained using disclosed embodiments of the present invention and DAB relative to a no primary control.

FIG. 46 compares FFPE tonsil, KRT15, red staining images (20×), hematoxylin counterstain, obtained using disclosed embodiments of the present invention and DAB images.

FIG. 47 compares FFPE tonsil, KRT15, blue staining images (20×), eosin counterstain, obtained using disclosed embodiments of the present invention and DAB images.

FIG. 48 compares FFPE tonsil, KRT15, blue staining images (20×), Methyl Green counterstain, obtained using disclosed embodiments of the present invention and DAB images.

FIG. 49 compares FFPE tonsil, Ki67, red staining images (20×), hematoxylin counterstain, obtained using disclosed embodiments of the present invention and DAB images.

FIG. 50 compares FFPE tonsil, Ki67, blue staining images (20×), Eosin counterstain, obtained using disclosed embodiments of the present invention and DAB images.

FIG. 51 compares FFPE tonsil, Ki67, red and blue staining images (20×), Methyl Green counterstain, obtained using disclosed embodiments of the present invention.

### DETAILED DESCRIPTION

### I. Abbreviations and Terms

### A. ABBREVIATIONS

AP: Ascorbate peroxidase
HRP: Horseradish peroxidase
IHC: Immunohistochemistry
ISH: In situ hybridization

### B. TERMS

The following explanations of terms are provided to better describe the present disclosure and to guide those of ordinary skill in the art in the practice of the present disclosure.

As used herein, “comprising” means “including” and the singular forms “a” or “an” or “the” include plural references unless the context clearly dictates otherwise.

The term “or” refers to a single element of stated alternative elements or a combination of two or more elements, unless the context clearly indicates otherwise.

Unless explained otherwise, all technical and scientific terms used herein have the same meaning as commonly understood to a person of ordinary skill in the art to which this disclosure pertains. Although methods and materials similar or equivalent to those described herein can be used to practice or test the present disclosure, suitable methods and materials are described below. The materials, methods, and examples are illustrative only and not intended to be limiting. Other features of the disclosure will be apparent to a person of ordinary skill in the art based on the detailed description and the claims.

The disclosure of numerical ranges should be understood as referring to each discrete point within the range, inclusive of endpoints, unless otherwise noted.

Unless otherwise indicated, all numbers expressing quantities of components, molecular weights, percentages, temperatures, times, and so forth, as used in the specification or claims are to be understood as being modified by the term “about.” Accordingly, unless otherwise implicitly or explicitly indicated, or unless the context is properly understood by a person of ordinary skill in the art to have a more definitive construction, the numerical parameters set forth are approximations that may depend on the desired properties sought and/or limits of detection under standard test conditions/methods as known to those of ordinary skill in the art. When directly and explicitly distinguishing embodiments from discussed prior art, the embodiment numbers are not approximates unless the word “about” is recited.

Although there are alternatives for various components, parameters, operating conditions, etc. set forth herein, that does not mean that those alternatives are necessarily equivalent and/or perform equally well. Nor does it mean that the alternatives are listed in a preferred order unless stated otherwise.

The presently disclosed compounds also include all isotopes of atoms present in the compounds, which can include, but are not limited to, deuterium, tritium, ¹⁸F, ¹⁴C, etc.

Definitions of common terms in chemistry may be found in Richard J. Lewis, Sr. (ed.), Hawley's Condensed Chemical Dictionary, published by John Wiley & Sons, Inc., 2016 (ISBN 978-1-118-13515-0).

Definitions of common terms in molecular biology may be found in Benjamin Lewin, Genes VII, published by Oxford University Press, 2000 (ISBN 019879276×); Kendrew et al. (eds.), The Encyclopedia of Molecular Biology, published by Blackwell Publishers, 1994 (ISBN 0632021829); and Robert A. Meyers (ed.), Molecular Biology and Biotechnology: a Comprehensive Desk Reference, published by Wiley, John & Sons, Inc., 1995 (ISBN 0471186341); and other similar references.

Absorbance: The retention by a compound or substance of certain wavelengths of radiation incident upon it; a measure of the amount of light at a particular wavelength absorbed as the light passes through a compound or substance, or through a solution of a compound or substance.

Absorption: The retention by a compound or substance of certain wavelengths of radiation incident upon it; a measure of the amount of light at a particular wavelength absorbed as the light passes through a compound or substance, or through a solution of a compound or substance.

Acyl: An organic functional group having the general formula —C(O)R, where R is hydrogen, alkyl, heteroalkyl, haloalkyl, aliphatic, heteroaliphatic, aryl, or heteroaryl.

Adsorption: The physical adherence or bonding of ions and molecules onto the surface of another molecule. An ion or molecule that adsorbs is referred to as an adsorbate. Adsorption can be characterized as chemisorption or physisorption, depending on the character and strength of the bond between the adsorbate and the substrate surface.

Alcohol: An organic compound including at least one hydroxyl (—OH) group. Alcohols may be monohydric (including one —OH group), dihydric (including two —OH groups; diols, such as glycols), trihydric (including three —OH; triols, such as glycerol) groups, or polyhydric (including three or more —OH groups; polyols). The organic portion of the alcohol may be aliphatic, cycloaliphatic (alicyclic), heteroaliphatic, cycloheteroaliphatic (heterocyclic), polycyclic, aryl, or heteroaryl, and may be substituted or unsubstituted.

Aliphatic: A substantially hydrocarbon-based compound, or a radical thereof (e.g., C₆H₁₃, for a hexane radical), including alkanes, alkenes, alkynes, including cyclic versions thereof, and further including straight- and branched-chain arrangements, and all stereo and position isomers as well. Unless expressly stated otherwise, an aliphatic group contains from one to twenty-five carbon atoms (C₁₋₂₅); for example, from one to fifteen (C₁₋₁₅), from one to ten (C₁₋₁₀), from one to six (C₁₋₆), or from one to four (C₁₋₄) carbon atoms. The term “lower aliphatic” refers to an aliphatic group containing from one to ten (C₁₋₁₀) carbon atoms. Unless expressly referred to as an “unsubstituted aliphatic,” an aliphatic group can either be unsubstituted or substituted. An aliphatic group can be substituted with one or more substituents (up to two substituents for each methylene carbon in an aliphatic chain, or up to one substituent for each carbon of a —C═C— double bond in an aliphatic chain, or up to one substituent for a carbon of a terminal methine group). Exemplary substituents include, but are not limited to, alkyl, alkenyl, alkynyl, alkoxy, alkylamino, alkylthio, acyl, aldehyde, amide, amino, aminoalkyl, aryl, arylalkyl, carboxyl, cyano, cycloalkyl, dialkylamino, halo, haloaliphatic, heteroaliphatic, heteroaryl, heterocycloaliphatic, hydroxyl, oxo, sulfonamide, sulfhydryl, thioalkoxy, or other functionality.

Alkoxy: A radical (or substituent) having the structure —OR, where R is a substituted or unsubstituted alkyl. Methoxy (—OCH₃) is an exemplary alkoxy group. In a substituted alkoxy, R is alkyl substituted with a non-interfering substituent. “Thioalkoxy” refers to —S—R, where R is substituted or unsubstituted alkyl. “Haloalkyloxy” means a radical —OR where R is a haloalkyl.

Alkyl: A hydrocarbon group having a saturated carbon chain. The chain may be cyclic, branched or unbranched. Examples, without limitation, of alkyl groups include methyl, ethyl, propyl, butyl, pentyl, hexyl, heptyl, octyl, nonyl and decyl. The term lower alkyl means the chain includes 1-10 carbon atoms. The terms alkenyl and alkynyl refer to hydrocarbon groups having carbon chains containing one or more double or triple bonds, respectively.

Alkylamino: A chemical functional group —N(H)R, where R is an alkyl group.

Alkylammonium: A cation having a formula └N(H)(R′)₃┘⁺ where each R′ independently is H or alkyl.

Amide: An organic compound characterized by a carbonyl group (C═O) linked to a nitrogen atom and having the following general formula, where R, R′ and R″ are the same or different, and typically are selected from hydrogen, aliphatic, and aryl.

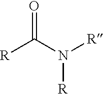

Amido: A chemical functional group —C(O)N(R)(R′) where R and R′ are independently hydrogen, alkyl, heteroalkyl, haloalkyl, aliphatic, heteroaliphatic, aryl (such as optionally substituted phenyl or benzyl), heteroaryl, alkylsulfano, or other functionality.

Amino: A chemical functional group —N(R)R′ where R and R′ are independently hydrogen, alkyl, heteroalkyl, haloalkyl, aliphatic, heteroaliphatic, aryl (such as optionally substituted phenyl or benzyl), heteroaryl, alkylsulfano, or other functionality. A “primary amino” group is —NH₂. “Mono-substituted amino” means a radical —N(H)R substituted as above and includes, e.g., methylamino, (l-methylethyl)amino, phenylamino, and the like. “Di-substituted amino” means a radical —N(R)R′ substituted as above and includes, e.g., dimethylamino, methylethylamino, di(1-methylethyl)amino, and the like.

Aminoalkyl: A chemical functional group —RNH₂ where R is an alkyl group.

Antibody: “Antibody” collectively refers to immunoglobulins or immunoglobulin-like molecules [including by way of example and without limitation. IgA, IgD, IgE, IgG and IgM, combinations thereof, and similar molecules produced during an immune response in any chordate such as a vertebrate, for example, in mammals such as humans, goats, rabbits and mice] and fragments thereof that specifically bind to a molecule of interest (or a group of highly similar molecules of interest) to the substantial exclusion of binding to other molecules. An “antibody” typically comprises a polypeptide ligand having at least a light chain or heavy chain immunoglobulin variable region that specifically recognizes and binds an epitope of an antigen. Immunoglobulins are composed of a heavy and a light chain, each of which has a variable region, termed the variable heavy (V_{H}) region and the variable light (V_{L}) region. Together, the V_{H} region and the V_{L} region are responsible for binding the antigen recognized by the immunoglobulin. Exemplary immunoglobulin fragments include, without limitation, proteolytic immunoglobulin fragments [such as F(ab′)₂ fragments. Fab′ fragments, Fab′-SH fragments and Fab fragments as are known in the art], recombinant immunoglobulin fragments (such as sFv fragments, dsFv fragments, bispecific sFv fragments, bispecific dsFv fragments, F(ab)′₂ fragments, single chain Fv proteins (“scFv”), and disulfide stabilized Fv proteins (“dsFv”). Other examples of antibodies include diabodies, and triabodies (as are known in the art), and camelid antibodies. “Antibody” also includes genetically engineered molecules, such as chimeric antibodies (for example, humanized murine antibodies), and heteroconjugate antibodies (such as, bispecific antibodies). See also, Pierce Catalog and Handbook, 1994-1995 (Pierce Chemical Co., Rockford, IL); Kuby, J., Immunology, 3^{rd} Ed., W.H. Freeman & Co., New York, 1997.

Aralkyl/arylalkyl: An aryl group (such as a phenyl group) appended to an alkyl radical including, but not limited to, benzyl, ethylbenzene, propylbenzene, butylbenzene, pentylbenzene, and the like. Conversely the term “phenylalkyl” refers to a phenyl group appended to an alkyl radical. Aralkyl groups, such as benzyl groups, may be unsubstituted or substituted with one, two or three substituents, with substituent(s) independently selected from alkyl, heteroalkyl, aliphatic, heteroaliphatic, thioalkoxy, haloalkyl (such as —CF₃), halo, nitro, cyano, —OR (where R is hydrogen or alkyl), —N(R)R′ (where R and R′ are independently of each other hydrogen or alkyl), —COOR (where R is hydrogen or alkyl) or —C(O)N(R′)R″ (where R′ and R″ are independently selected from hydrogen or alkyl). Non-limiting examples, include o-, in-, and/or p-chlorobenzyl, o-, m-, and/or p-methoxybenzyl, and o-, m-, and/or p-(trifluoromethyl)benzyl.

Aromatic or aryl: Unsaturated, cyclic hydrocarbons having alternate single and double bonds. An aromatic system may have a single ring, such as benzene, a 6-carbon ring containing three double bonds, or the aromatic compound may have multiple rings, such as fused ring systems, including naphthalene, anthracene, chrysene, or phenanthrene.

Aryl: A monovalent aromatic carbocyclic group of, unless specified otherwise, from 6 to 20 carbon atoms having a single ring (e.g., phenyl) or multiple condensed rings in which at least one ring is aromatic (e.g., quinoline, indole, benzodioxole, and the like), provided that the point of attachment is through an atom of an aromatic portion of the aryl group and the aromatic portion at the point of attachment contains only carbons in the aromatic ring. If any aromatic ring portion contains a heteroatom, the group is a heteroaryl and not an aryl. Aryl groups are monocyclic, bicyclic, tricyclic or tetracyclic.

Arylalkyl: An acyclic alkyl group in which one of the hydrogen atoms bonded to a carbon atom, typically a terminal or sp³ carbon atom, is replaced with an aryl group. Typical arylalkyl groups include, but are not limited to, benzyl, 2-phenylethan-1-yl, naphthylmethyl, 2-naphthylethan-1-yl, naphthobenzyl, 2-naphthophenylethan-1-yl and the like. Where specific alkyl moieties are intended, the nomenclature arylalkanyl, arylalkenyl and/or arylalkynyl may be used.

Biomolecule: Any molecule that may be included in a biological system, including but not limited to, a synthetic or naturally occurring protein, glycoprotein, lipoprotein, amino acid, nucleoside, nucleotide, nucleic acid, oligonucleotide, DNA, RNA, carbohydrate, sugar, lipid, fatty acid, hapten, and the like.

Carbonyl: A radical of the formula —C(O)—. Carbonyl-containing groups include any substituent containing a carbon-oxygen double bond (C═O), including acyl groups, amides, carboxy groups, esters, ureas, carbamates, carbonates and ketones and aldehydes, such as substituents based on —COR or —RCHO where R is an aliphatic, heteroaliphatic, alkyl, heteroalkyl, hydroxyl, or a secondary, tertiary, or quaternary amine.

Chemifluorescence: Fluorescence that occurs when a chemical reaction converts a non-fluorescent reactant into a product that fluoresces after exposure to an excitation source.

Chemisorption: A type of adsorption characterized by a strong interaction between an adsorbate and a substrate. Strong interactions include formation of covalent and/or ionic bonds, thus changing the electronic structure of the adsorbate. Chemisorption can be reversed with high temperature or a chemical reaction that breaks the bond between the adsorbate and the substrate.

Chromophore: An aromatic compound comprising a chemical grouping that gives color to the compound by causing displacement of, or appearance of, absorbent bands in the visible spectrum. Exemplary chromophores include, but are not limited to, R—N═N— (e.g., azo dyes).

Chromogen: A chemical compound that can, by chemical or other means, be converted into a chromophore. Exemplary chromogens include, but are not limited to, naphthols, aryl diazonium salts, and 1,3-diketones.

Conditions sufficient to detect: Any environment that permits the desired activity, for example, that permits a probe to bind a target and the interaction to be detected. For example, such conditions include appropriate temperatures, buffer solutions, and detection means such as microscopes and digital imaging equipment.

Conjugate: Two or more moieties directly or indirectly coupled together. For example, a first moiety may be covalently or noncovalently (e.g., electrostatically) coupled to a second moiety. Indirect attachment is possible, such as by using a “linker” (a molecule or group of atoms positioned between two moieties).

Contacting: Placement that allows association between two or more moieties, particularly direct physical association, for example both in solid form and/or in liquid form (for example, the placement of a biological sample, such as a biological sample affixed to a slide, in contact with a composition, such as a solution containing the compositions disclosed herein).

Conjugating, joining, bonding or linking: Coupling a first unit to a second unit. This includes, but is not limited to, covalently bonding one molecule to another molecule, noncovalently bonding one molecule to another (e.g. electrostatically bonding) (see, for example, U.S. Pat. No. 6,921,496, which discloses methods for electrostatic conjugation), non-covalently bonding one molecule to another molecule by hydrogen bonding, non-covalently bonding one molecule to another molecule by van der Waals forces, and any and all combinations of such couplings.

Counterstaining: A method of post-treating samples after they have already been stained with agents to detect one or more targets, such that their structures can be more readily visualized. For example, a counterstain is optionally used prior to coverslipping to render a IHC stain more distinct. Counterstains differ in color from a primary stain. Numerous counterstains are well known, such as hematoxylin, eosin, methyl green, methylene blue, Giemsa, Alcian blue, DAPI, and Nuclear Fast Red. In some examples, more than one stain can be mixed together to produce the counterstain. This provides flexibility and the ability to choose stains. For example, a first stain, can be selected for the mixture that has a particular attribute, but yet does not have a different desired attribute. A second stain can be added to the mixture that displays the missing desired attribute. For example, toluidine blue, DAPI, and pontamine sky blue can be mixed together to form a counterstain.

Coupled: The term “coupled” means joined together, either directly or indirectly. A first atom or molecule can be directly coupled or indirectly coupled to a second atom or molecule. A secondary antibody provides an example of indirect coupling. One specific example of indirect coupling is a rabbit anti-hapten primary antibody that is bound by a mouse anti-rabbit IgG antibody, which is in turn bound by a goat anti-mouse IgG antibody that is covalently linked to a detectable label.

Detect: To determine if an agent (such as a signal or particular antigen, protein or nucleic acid) is present or absent, for example, in a sample. In some examples, this can further include quantification, and/or localization, for example localization within a cell or particular cellular compartment. “Detecting” refers to any method of determining if something exists, or does not exist, such as determining if a target molecule is present in a biological sample. For example, “detecting” can include using a visual or a mechanical device to determine if a sample displays a specific characteristic. In certain examples, detection refers to visually observing a probe bound to a target, or observing that a probe does not bind to a target. For example, light microscopy and other microscopic means are commonly used to detect chromogenic precipitates for methods described here.

Detectable Label: A molecule or material that can produce a detectable (such as visually, electronically or otherwise) signal that indicates the presence and/or concentration of a target, such as a target molecule, in a sample, such as a tissue sample. When conjugated to a specific binding molecule, the detectable label can be used to locate and/or quantify the target to which the specific binding molecule is directed. Thereby, the presence and/or concentration of the target in a sample can be detected by detecting the signal produced by the detectable label. A detectable label can be detected directly or indirectly, and several different detectable labels conjugated to different specific-binding molecules can be used in combination to detect one or more targets. Multiple detectable labels that can be separately detected can be conjugated to different specific binding molecules that specifically bind different targets to provide a multiplexed assay that can provide detection of the multiple targets in a sample. Detectable labels include, but are not limited to colored, fluorescent, phosphorescent and luminescent molecules and materials, and more particularly for the present disclosure concern colored and/or fluorescent labels.

Emission or emission signal: The light of a particular wavelength generated from a source. In particular examples, an emission signal is emitted from a fluorophore after the fluorophore absorbs light at its excitation wavelength(s).

Excitation or excitation signal: The light of a particular wavelength necessary and/or sufficient to excite an electron transition to a higher energy level. In particular examples, an excitation is the light of a particular wavelength necessary and/or sufficient to excite a fluorophore to a state such that the fluorophore will emit a different (such as a longer) wavelength of light than the wavelength of light from the excitation signal.

Fixation: A process which preserves cells and tissue constituents in as close to a life-like state as possible and allows them to undergo preparative procedures without change. Fixation arrests the autolysis and bacterial decomposition processes that begin upon cell death, and stabilizes the cellular and tissue constituents so that they withstand the subsequent stages of tissue processing, such as for IHC.

Tissues may be fixed by either perfusion with or submersion in a fixative, such as an aldehyde (such as formaldehyde, paraformaldehyde, glutaraldehyde, and the like). Other fixatives include oxidizing agents (for example, metallic ions and complexes, such as osmium tetroxide and chromic acid), protein-denaturing agents (for example, acetic acid, methanol, and ethanol), fixatives of unknown mechanism (for example, mercuric chloride, acetone, and picric acid), combination reagents (for example, Carnoy's fixative, methacarn, Bouin's fluid, B5 fixative, Rossman's fluid, and Gendre's fluid), microwaves, and miscellaneous (for example, excluded volume fixation and vapour fixation). Additives also may be included in the fixative, such as buffers, detergents, tannic acid, phenol, metal salts (for example, zinc chloride, zinc sulfate, and lithium salts), and lanthanum. A common fixative for preparing samples for IHC is formaldehyde, generally in the form of a formalin solution (4% formaldehyde in a buffer solution, referred to as 10% buffered formalin).

Fluorescence: The emission of visible radiation by an atom or molecule passing from a higher to a lower electronic state, wherein the time interval between absorption and emission of energy is 10⁻⁸ to 10⁻³ second. Fluorescence occurs when the atom or molecule absorbs energy from an excitation source (e.g., an ultraviolet lamp) and then emits the energy as visible radiation.

Fluorophore: The functional group, or portion, of a molecule that causes the molecule to fluoresce when exposed to an excitation source. The term “fluorophore” also is used to refer to fluorescent compounds used as dyes to mark proteins with a fluorescent label.

Fluorogen: A chemical compound that can, by chemical or other means, be converted into a fluorophore. Exemplary fluorogens include, but are not limited to, the leuco forms of common fluorophores such as rhodamines and fluoresceins.

Functional group: A specific group of atoms within a molecule that is responsible for the characteristic chemical reactions of the molecule. Exemplary functional groups include, without limitation, alkyl, alkenyl, alkynyl, aryl, halo (fluoro, chloro, bromo, iodo), epoxide, hydroxyl, carbonyl (ketone), aldehyde, carbonate ester, carboxylate, carboxyl, ether, ester, peroxy, hydroperoxy, carboxamide, amino (primary, secondary, tertiary), ammonium, imide, azide, cyanate, isocyanate, thiocyanate, nitrate, nitrite, nitrile, nitroalkyl, nitroso, pyridyl, phosphate, sulfonyl, sulfide, thiol (sulfhydryl), disulfide.

Halogen: Flourine, bromine, chloring or iodine.

Hapten: A molecule, typically a small molecule that can combine specifically with an antibody, but typically is substantially incapable of being immunogenic except in combination with a carrier molecule. Examples of haptens include, but are not limited to fluorescein, biotin, nitroaryls, including, but, not limited to, dinitrophenol (DNP), digoxigenin, oxazole, pyrazole, thiazole, benzofuran, urea, thiourca, rotenoid, coumarin and cyclolignan.

Heteroaryl: An aromatic compound or group having at least one heteroatom, i.e., one or more carbon atoms in the ring has been replaced with an atom having at least one lone pair of electrons, typically nitrogen, oxygen, phosphorus, silicon, or sulfur.

Hydrocarbon: An organic compound consisting of the elements carbon and hydrogen. Hydrocarbons typically are derived from petroleum, coal tar, and plant sources. Hydrocarbons include aliphatic compounds (alkanes, alkenes, alkynes, and cyclic versions thereof, including straight- and branched-chain arrangements), aromatic compounds (unsaturated, cyclic hydrocarbons having alternate single and double bonds), and combinations thereof (e.g., arylalkyl compounds).

Immunohistochemistry (IHC): A method of determining the presence or distribution of an antigen in a sample by detecting interaction of the antigen with a specific binding agent or moiety, such as an antibody. A sample including an antigen (such as a target antigen) is incubated with an antibody under conditions permitting antibody-antigen binding. Antibody-antigen binding can be detected by means of a detectable label conjugated to the antibody (direct detection) or by means of a detectable label conjugated to a secondary antibody, which is raised against the primary antibody (e.g., indirect detection). Detectable labels include, but are not limited to, radioactive isotopes, fluorochromes (such as fluorescein, fluorescein isothiocyanate, and rhodamine), enzymes and chromogenic molecules.

Lower: Refers to organic compounds having 10 or fewer carbon atoms in a chain, including all branched and stereochemical variations, particularly including methyl, ethyl, propyl, butyl, pentyl, hexyl, heptyl, octyl, nonyl, and decyl.

Moiety: A moiety is a fragment of a molecule, or a portion of a conjugate.

Molecule of interest or target molecule: A molecule for which the presence, location and/or concentration is to be determined. Examples of molecules of interest include proteins and nucleic acid sequences present in tissue samples.

Multiplex, -ed, -ing: Detection of multiple targets in a sample substantially simultaneously, or sequentially, as desired, using plural different conjugates. Multiplexing can include identifying and/or quantifying nucleic acids generally. DNA, RNA, peptides, proteins, both individually and in any and all combinations. Multiplexing also can include detecting two or more of a gene, a messenger and a protein in a cell in its anatomic context.

Polypeptide: A polymer in which the monomers are amino acid residues that are joined together through amide bonds. When the amino acids are alpha-amino acids, either the L-optical isomer or the D-optical isomer can be used. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced. The term “residue” or “amino acid residue” includes reference to an amino acid that is incorporated into a protein, polypeptide, or peptide.

Probe: A substance used to detect or identify another substance in a sample.

Sample: The term “sample” refers to any liquid, semi-solid or solid substance (or material) in or on which a target can be present. In particular, a sample can be a biological sample or a sample obtained from a biological material. A biological sample is any solid or fluid sample obtained from, excreted by or secreted by any living organism, including without limitation, single celled organisms, such as bacteria, yeast, protozoans, and amoebas among others, multicellular organisms (such as plants or animals, including samples from a healthy or apparently healthy human subject or a human patient affected by a condition or disease to be diagnosed or investigated, such as cancer). For example, a biological sample can be a biological fluid obtained from, for example, blood, plasma, serum, urine, bile, ascites, saliva, cerebrospinal fluid, aqueous or vitreous humor, or any bodily secretion, a transudate, an exudate (for example, fluid obtained from an abscess or any other site of infection or inflammation), or fluid obtained from a joint (for example, a normal joint or a joint affected by disease). A biological sample can also be a sample obtained from any organ or tissue (including a biopsy or autopsy specimen, such as a tumor biopsy) or can include a cell (whether a primary cell or cultured cell) or medium conditioned by any cell, tissue or organ. In some examples, a biological sample is a nuclear extract. In some examples, a biological sample is bacterial cytoplasm. In other examples, a sample is a test sample. For example, a test sample is a cell, a tissue or cell pellet section prepared from a biological sample obtained from a subject. In an example, the subject is one that is at risk or has acquired a particular condition or disease.

Section: A section of a tissue sample refers to a single part or piece of a tissue sample, such as a thin slice of tissue or cells cut from a tissue sample. Multiple sections of tissue samples may be taken and subjected to analysis according to the present invention. The section can be as small as one cell or two cells, or many thousands of cells.

Specific binding moiety or specific binding agent: A member of a specific-binding pair. Specific binding pairs are pairs of molecules that are characterized in that they bind each other to the substantial exclusion of binding to other molecules (for example, specific binding pairs can have a binding constant that is at least 10³ M⁻¹ greater, 10⁴ M⁻¹ greater or 10⁵ M⁻¹ greater than a binding constant for either of the two members of the binding pair with other molecules in a biological sample). Particular examples of specific binding moieties include specific binding proteins (for example, antibodies, lectins, avidins such as streptavidins, and protein A), nucleic acids sequences, and protein-nucleic acids. Specific binding moieties can also include the molecules (or portions thereof) that are specifically bound by such specific binding proteins.

Specifically binds: A term that refers to the binding of agent that preferentially binds to a defined target (such as an antibody to a specific antigen or a nucleic acid probe to a specific nucleic acid sequence). With respect to an antigen, “specifically binds” refers to the preferential association of an antibody or other ligand, in whole or part, with a specific polypeptide. With respect to a nucleic acid sequence, “specifically binds” refers to the preferential association of a nucleic acid probe, in whole or part, with a specific nucleic acid sequence

A specific binding agent or moiety binds substantially only to a defined target. It is recognized that a minor degree of non-specific interaction may occur between a molecule, such as a specific binding agent or moiety, and a non-target polypeptide or non-target nucleic acid sequence. Although a selectively reactive antibody binds an antigen, it can do so with low affinity. Antibody to antigen specific binding typically results in greater than 2-fold, such as greater than 5-fold, greater than 10-fold, or greater than 100-fold increase in amount of bound antibody or other ligand (per unit time) to a target polypeptide, as compared to a non-target polypeptide. A variety of immunoassay formats are appropriate for selecting antibodies specifically immunoreactive with a particular protein. For example, solid-phase ELISA immunoassays are routinely used to select monoclonal antibodies specifically immunoreactive with a protein. See Harlow & Lane, Antibodies, A Laboratory Manual, Cold Spring Harbor Publications, New York (1988), for a description of immunoassay formats and conditions that can be used to determine specific immunoreactivity.

Nucleic acid probe to nucleic acid sequence specific binding typically results in greater than 2-fold, such as greater than 5-fold, greater than 10-fold, or greater than 100-fold increase in amount of bound nucleic acid probe to a target nucleic acid sequence, as compared to a non-target nucleic acid. A variety of ISH conditions are appropriate for selecting nucleic acid probes that bind specifically with a particular nucleic acid sequence.

Stain: Any biological or chemical entity which, when applied to targeted molecules in biological sample, renders the molecules detectable under microscopic examination. Stains include, without limitation, detectable nucleic acid probes, antibodies, dyes and other reagents which in combination or by themselves result in a colored end product (by bright field or fluorescence detection methodologies). “staining” or “microscopic visualization” may be performed by any means, including, but not limited to, immunostaining that involves antibody detection of a desired antigen, followed by an enzymatic reaction yielding a color reaction product, and may involve computerized image analysis.

Substituent: An atom or group of atoms that replaces another atom in a molecule as the result of a reaction. The term “substituent” typically refers to an atom or group of atoms that replaces a hydrogen atom, or two hydrogen atoms if the substituent is attached via a double bond, on a parent hydrocarbon chain or ring. The term “substituent” may also cover groups of atoms having multiple points of attachment to the molecule, e.g., the substituent replaces two or more hydrogen atoms on a parent hydrocarbon chain or ring. In such instances, the substituent, unless otherwise specified, may be attached in any spatial orientation to the parent hydrocarbon chain or ring. Exemplary substituents include, for instance, alkyl, alkenyl, alkynyl, alkoxy, alkylamino, alkylthio, acyl, aldehyde, amido, amino, aminoalkyl, aryl, arylalkyl, arylamino, carbonate, carboxyl, cyano, cycloalkyl, dialkylamino, halo, haloaliphatic (e.g., haloalkyl), haloalkoxy, heteroaliphatic, heteroaryl, heterocycloaliphatic, hydroxyl, oxo, sulfonamide, sulfhydryl, thio, and thioalkoxy groups.

Substituted: A fundamental compound, such as an aryl or aliphatic compound, or a radical thereof, having coupled thereto one or more substituents, each substituent typically replacing a hydrogen atom on the fundamental compound. A person of ordinary skill in the art will recognize that compounds disclosed herein may be described with reference to particular structures and substituents coupled to such structures, and that such structures and/or substituents also can be further substituted, unless expressly stated otherwise or context dictates otherwise. Solely by way of example and without limitation, a substituted aryl compound may have an aliphatic group coupled to the closed ring of the aryl base, such as with toluene. Again solely by way of example and without limitation, a long-chain hydrocarbon may have a hydroxyl group bonded thereto.

Tissue: A collection of interconnected cells that perform a similar function within an organism. Any collection of cells that can be mounted on a standard glass microscope slide including, without limitation, sections of organs, tumor sections, bodily fluids, smears, frozen sections, cytology preps, and cell lines.

Tyramine: A compound having the formula C₈H₁₁NO, also known as 4-(2-aminoethyl)phenol.

Tyramide: A tyramine derivative, wherein the amine functional group of a tyramine molecule has formed an amide bond with a carbonyl-containing functional group.

### II. Introduction

Certain disclosed embodiments concern chromogens that are attached to tyramide covalent linker substrates to provide an uncolored covalent label that can be deposited at or around areas of interest by HRP enzymatic reaction in cell or tissue samples. After deposition, color is then developed by the addition of different agents, such as diazonium salts, that react with the pro-chromogens deposited at or about the target to form colored compounds at or around the areas of interest, that can be detected by brightfield, fluorescent microscopy, or both. Since the label is bound covalently, it is more stable than classic precipitating substrates for use with either aqueous or organic mountants. Since the coloring agent, such as diazonium salts, are coupled to the chromogen subsequent to covalent labeling of a target, a single chromogen substrate can be used to generate multiple detectable colors during an IHC process. Disclosed covalent chromogenic substrates provide (1) better signal stability in different mounting media, and (2) tighter staining patterns

### III. Compounds

Certain embodiments of the present invention concern using compounds satisfying Formula I, below 7

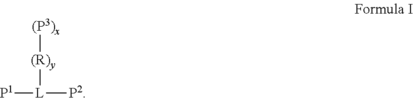

With reference to Formula I, P¹, P² and P³ are aromatic rings or aromatic ring systems, such as fused ring systems. The P and P³ ring systems can be the same or different and each may comprise at least two fused rings, wherein at least one of the fused rings comprises a structure represented as

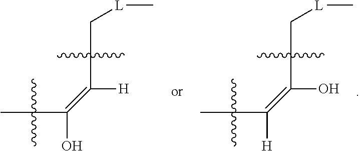

P² is a second aromatic ring system, typically substituted with at least one —OH group. L is a linker having from 1 to at least 50 atoms, more typically from 2 to 20 atoms in a chain. L can be substituted or unsubstituted. L can be, for example, substituted with a P³ group if no R group is present, or can be substituted with R—P³. R is a bond, or is independently an L group. x and y independently are 0 to 5, with certain disclosed embodiments having x and y be 0 or 1.

Certain disclosed embodiments of the present invention may satisfy a Formula II

P¹-L-P² Formula II,

where P¹, P² and L are as stated for Formula I.

Aromatic ring systems according to the present invention may be either hydrocarbon ring systems or heteroaryl ring systems. For certain embodiments, P¹ and P³ are aromatic hydrocarbon fused ring systems, such as a fused ring system having from 2 to at least 4 rings in a fused-ring system. P¹ and/or P³ may be substituted with at least one solubility moiety selected to increase the solubility of the compound in solvents, such as aqueous solvent systems, commonly used for staining protocols, such as IHC. Representative P¹ and P³ fused-ring systems include, but are not limited to:

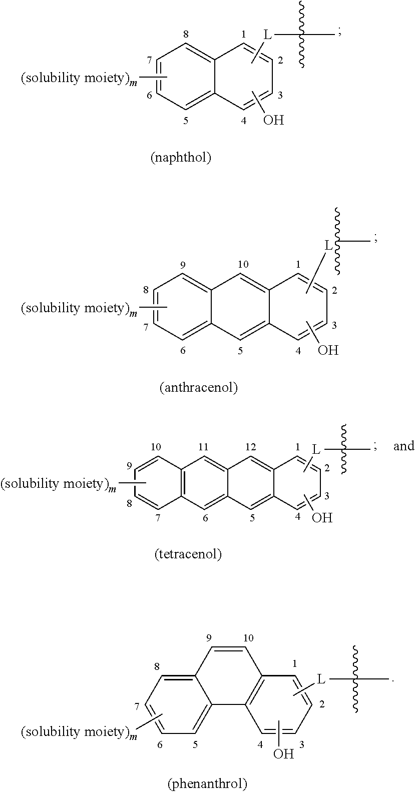

With reference to these exemplary P¹ and P³ fused-ring systems, m is from 1 to 5, more typically 1-3, with certain disclosed embodiments having in =1. Representative “solubility moieties,” particularly aqueous solubility enhancer moieties, can be selected from —SO₃⁻, —SO₃H, —CO₂⁻, —CO₂H, —HPO₃⁻², H₂PO₃⁻¹, H₃PO₃, PEG,

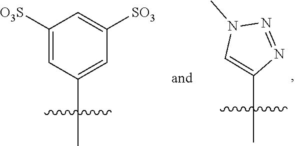

and salt forms thereof.

As indicated, P¹ and/or P³ may include at least one hydroxyl (—OH) group on the ring system. The at least one hydroxyl group typically is ortho or meta to L. Accordingly, certain disclosed compounds have a following characteristic: P¹ and/or P³ is naphthol, L is coupled to position 2 or 3, at least one hydroxyl group is at positions 3 and/or 4, and the at least one moiety selected to increase the solubility of the compound, such as —SO₃⁻ or a salt form thereof, is coupled to at least one of the 5-8 positions; P¹ and/or P³ is anthracenol, L is coupled to position 2 or 3, at least one hydroxyl group is at positions 3 and/or 4, and the at least one moiety selected to increase the solubility of the compound is coupled to at least one of the 6-9 positions; P¹ and/or P³ is tetracenol, L is coupled to position 2 or 3, at least one hydroxyl group is at positions 3 and/or 4, and the at least one moiety selected to increase the aqueous solubility of the compound is coupled to at least one of the 7-10 positions; or P¹ and/or P³ is phenanthrol, L is coupled to position 2 or 3, at least one hydroxyl group is at positions 3 and/or 4, and the at least one moiety selected to increase the solubility of the compound is coupled to at least one of the 5-8 positions.

P¹ and/or P³ may be a heteroaryl fused ring system, typically a heteroaryl fused ring system that is electron rich. Examples, without limitation, of suitable heteroaryl fused ring systems, include fused furan, fused thiophene, and fused pyrrole ring systems, such as

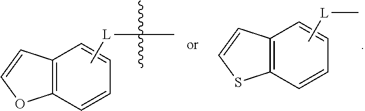

P² may be an aromatic hydrocarbon. P² for certain disclosed embodiments comprises a phenol, and even more particular examples of P² comprise a phenol where the hydroxyl group is para to L.

L is a covalent bond, or is selected from C₁-C₅₀ aliphatic, C₁-C₅₀ alkyl, C₁-C₅₀ alkyl amide. C₁-C₅₀ alkyl ester, C₁-C₅₀ ketone, a C₂-C₅₀ polyol, including glycols, C₂-C₅₀ polyalkyl ethers, such as polyalkylene oxide, including by way of example a PEG linker, and C₂-C₅₀ aliphatic amines. The C₁-C₅₀ linker length for certain embodiments is more typically C₁-C₂₀. For particular disclosed embodiments, L is an alkyl amide, such as

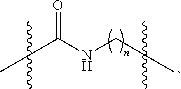

where n is from 1-10, more typically 1-5, such as 2. As used herein, wavy lines ( [symbol] ) indicate points of attachment to other portions of the compounds, such as to P¹ and P². A particular example of L is

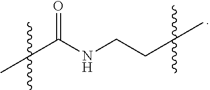 [SMILES: *CCNC(*)=O]

For certain disclosed embodiments, L is an ether or a polyether designated as

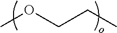

where o designates the number of ethoxy units and is from 1 to 10, and more typically 3 to 5. A person of ordinary skill in the art will appreciate that other alkoxy units, such as propoxy and butoxy ethers, also can form suitable linkers. Such polyalkoxylated groups can be connected to aromatic rings or ring systems using any acceptable functional group as will be understood by a person of ordinary skill in the art, with certain disclosed embodiments coupling L groups to aromatic rings or ring systems using amides, amines or esters, as exemplified by the following linker,

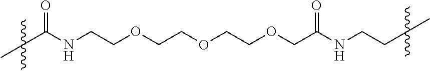 [SMILES: *CCNC(=O)COCCOCCOCCNC(*)=O]

where again the wavy lines indicate points of attachment to P¹, P² and P³ aromatic rings or aromatic ring systems. Yet additional embodiments concern aliphatic amine linkers, exemplified by

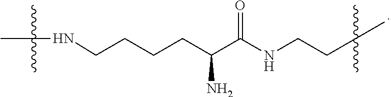 [SMILES: *CCNC(=O)[C@@H](N)CCCCN*]

Certain disclosed embodiments of compounds useful for the present invention satisfying Formulas I and/or II comprise: a P¹ that is selected from:

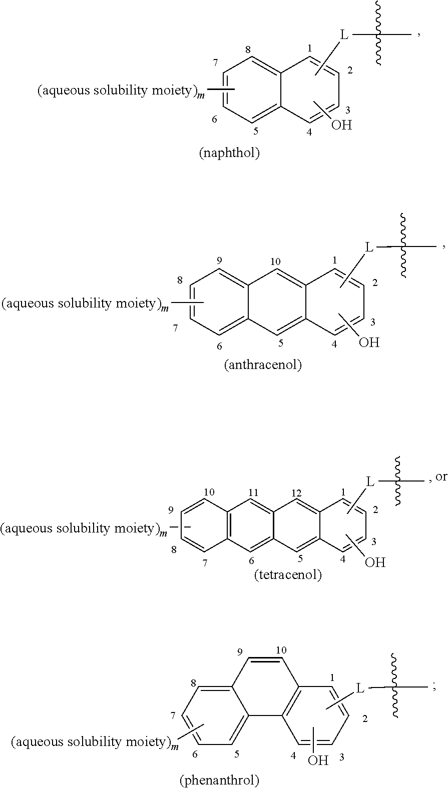

m is from 1 to 3; P² comprises a phenol; the at least one solubility moiety is selected to increase the aqueous solubility of the compound and is selected from —SO₃⁻, —SO₃H, —CO₂⁻, —CO₂H, —HPO₃⁻², H₂PO₃⁻¹, H₃PO₃, PEG,

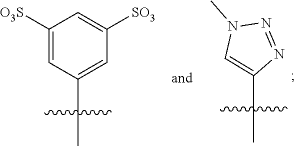

and L is selected from a covalent bond, C₁-C₂₀ alkyl, a C₁-C₂₀ alkyl amide, a C₁-C₁₀ ketone, or a C₁-C₁₀ glycol or polyol.

Disclosed embodiments also may have a Formula III, below

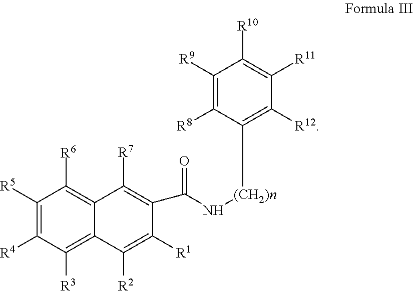

With reference to Formula III, R¹ and R² are independently selected from H and OH; R³-R⁸ are independently selected from H, —F, —Cl, —Br, —I, —OH, and —OR⁹ where R⁹ is C₁-C₆ alkyl, —SO₃⁻, —SO₃H, —CO₂⁻, —CO₂H, —HPO₃⁻², H₂PO₃⁻¹, H₃PO₃, PEG,

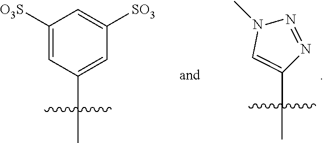

At least one of R¹, R², R⁷, and at least one of R⁸-R¹², is hydroxyl. Alternatively, R³ and R⁴ taken together form at least one fused ring system; and/or R⁴ and R₅ taken together form at least one fused ring system; and/or R⁵ and R⁶ taken together form at least one fused ring system; and n is 1 to 5. For particular compounds if R¹ is H, then R² is OH, and if R¹ is OH, then R₂ is H; and R³, R⁴, R⁵, and R⁶ are independently H or —SO₃⁻, with at least one of R³, R⁴, R⁵, or R⁶ being —SO₃⁻, or a salt form thereof.

One example of a compound satisfying Formula I above, shown without positively charged counterions, such as Na⁺ metal counterions, is

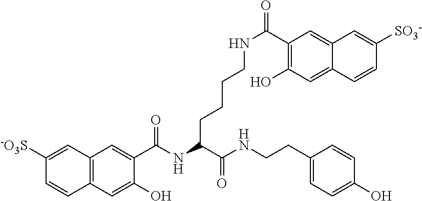

### (R)-7,7′-(((6-((4-hydroxyphenethyl)amino)-6-oxohexane-1,5-diyl)bis(azanediyl))bis(carbonyl))bis(6-hydroxynaphthalene-2-sulfonate)

An example of a compound satisfying Formulas I-III, again shown without a counterion, is

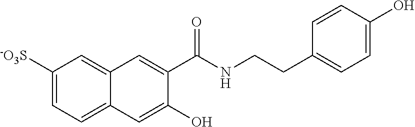

### 6-hydroxy-7-((4-hydroxyphenethyl)carbamoyl)naphthalene-2-sulfonate

Compounds exemplifying the P1, P2, L and R features of the general formulas discussed above are provided in Table I below.

TABLE 1

| | |
|---|---|
| 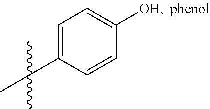 [SMILES: *c1ccc(O)cc1] | P² |
| 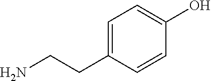 | L-P² with both P² and L provided by tyramine. |
| 4-(2-aminoethyl)phenol (tyramine) | |
| 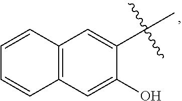 [SMILES: *c1cc2ccccc2cc1O] | P¹ |
| 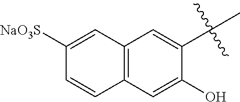 | P¹ + a solubility enhancer. |
| 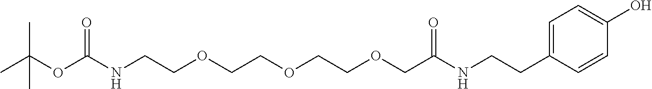 [SMILES: CC(C)(C)OC(=O)NCCOCCOCCOCC(=O)NCCc1ccc(O)cc1] | L-P² with a t-BOC protected linker. |
| tert-butyl (1-(4-hydroxyphenyl)-4-oxo-6,9,12-trioxa-3-azatetradecan-14- | |
| yl)carbamate | |
| 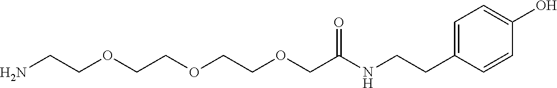 [SMILES: NCCOCCOCCOCC(=O)NCCc1ccc(O)cc1] | L-P² |
| 2-(2-(2-(2-aminoethoxy)ethoxy)ethoxy)-N-(4-hydroxyphenethyl)acetamide | |
| 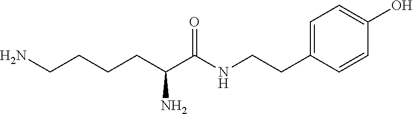 [SMILES: NCCCC[C@H](N)C(=O)NCCc1ccc(O)cc1] | L-P² |
| (S)-2,6-diamino-N-(4-hydroxyphenethyl)hexanamide | |
| 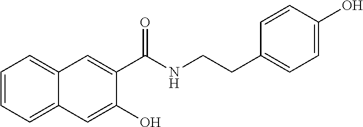 [SMILES: O=C(NCCc1ccc(O)cc1)c1cc2ccccc2cc1O] | P¹-L-P² |
| 6-hydroxy-7-((4-hydroxyphenethyl)carbamoyl)naphthalene | |
| 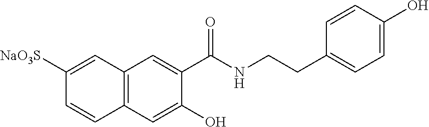 | P¹ with a solubility enhancer-L- P² |
| sodium 6-hydroxy-7-((4-hydroxyphenethyl)carbamoyl)naphthalene- | |
| 2-sulfonate | |
| 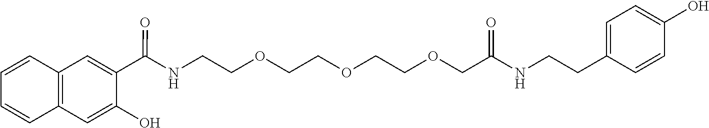 [SMILES: O=C(COCCOCCOCCNC(=O)c1cc2ccccc2cc1O)NCCc1ccc(O)cc1] | P¹-L-P² |
| 3-hydroxy-N-(1-(4-hydroxyphenyl)-4-oxo-6,9,12-trioxa-3-azatetradecan-14- | |
| yl)-2-naphthamide | |
| 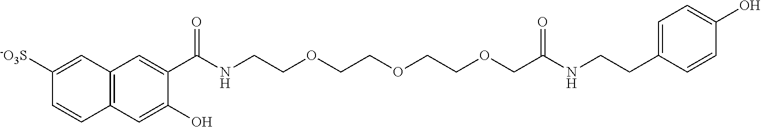 | P¹ with a solubility enhancer-L- P² |
| 3-hydroxy-N-(1-(4-hydroxyphcnyl)-4-oxo-6,9,12-trioxa-3-azatetradccan-14- | |
| y1)-2-naphthamide sulfonate | |
| 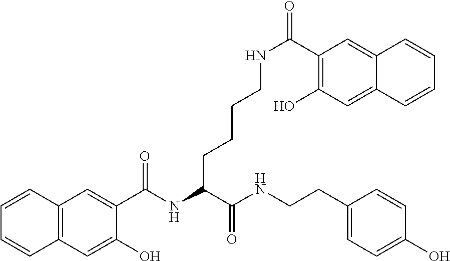 | 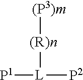 |
| (R)-7,7′-(((6-((4-hydroxyphenethyl)amino)-6-oxohexane-1,5- | |
| diyl)bis(azanediyl))bis(carbonyl))bis(6-hydroxynaphthalene) | |
| 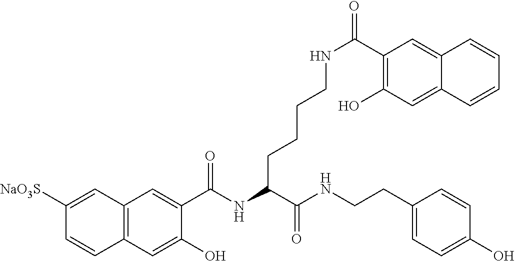 | 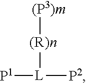 with P¹ comprising a solubility enhancer |
| 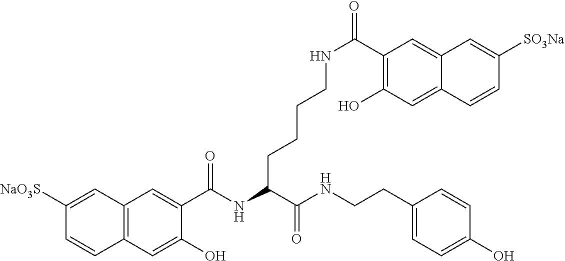 | 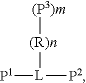 with both P¹ and P³ comprising a solubility enhancer |
| sodium (R)-7,7'-(((6-((4-hydroxyphenethyl)amino)-6-oxohexane-1,5- | |
| diyl)bis(azanediyl))bis(carbonyl))bis(6-hydroxynaphthalene-2-sulfonate) | |

Compounds according to Formulas I-III can be used to label targets in biological sample. Accordingly, the present application also concerns covalent conjugates comprising a biological sample comprising a target and at least one compound according to Formulas I. II and/or III covalently associated with the target, as illustrated below by Formula IV.

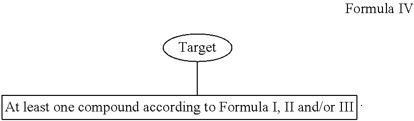

A specific example of a tyramide-based conjugate satisfying Formula IV is provided below as Formula V.

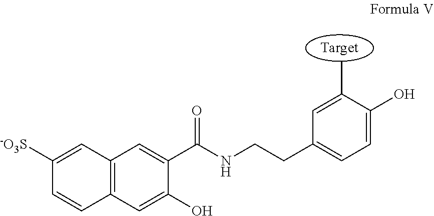

Compounds according to Formula I-III can be reacted with a chromogen and/or fluorogen to provide a conjugate that can be visualized, such as by using brightfield and/or fluorescent microscopy. Accordingly, the present application also includes compounds satisfying Formulas I, II and/or III that are coupled to a chromogen and/or a fluorogen, as indicated below by Formula VI,

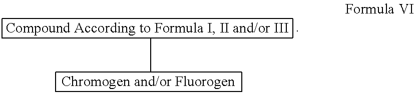

A more particular example of a tyramide-based conjugate is provided below as Formula VII, where “ring” A refers to an aromatic system as defined above, and the “aqueous solubility moiety” also is as defined above.

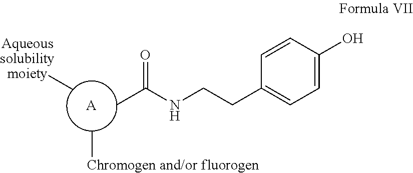

Azo dyes provide particular examples of chromophores and/or fluorophores. And/or in this context indicates that certain azo dyes can produce a particular color for viewing by, for example, brightfield microscopy, and also can be a fluorescing agent suitable for viewing using fluorescent microscopy. Again with specific reference to tyramide compounds as examples, such tyramide-based compounds may have the following general Formula VIII

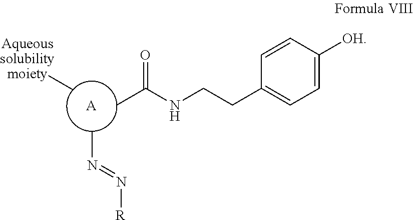

Examples of particular azo dye conjugates are discussed in more detail below with reference to embodiments of a method for using such compounds.

Conjugates comprising at least one compound according to the present invention coupled to a biological target, and further coupled to a chromogen and/or fluorogen, such as an azo dye, also are within the scope of the present invention. Such compounds are exemplified as Formula IX, below

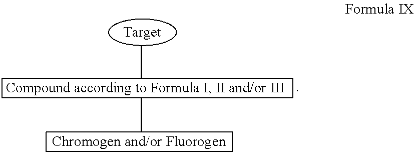

Tyramide-based compounds coupled to both a biological target and an azo dye chromophore/fluorophore may have the following Formula X

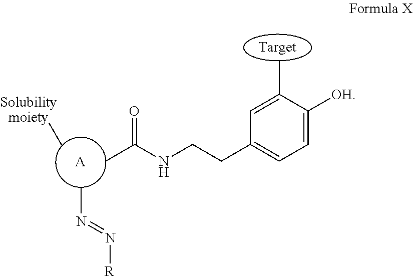

### IV. Synthesis of Compounds Satisfying Formulas I, II and III

Compounds satisfying Formulas I and II can be made by any suitable synthetic protocol. One example of a method to make compounds according to Formulas I and II is provided below as Scheme I. Additional details concerning synthesis of compounds according to Formulas I and II are provided below in the Examples.

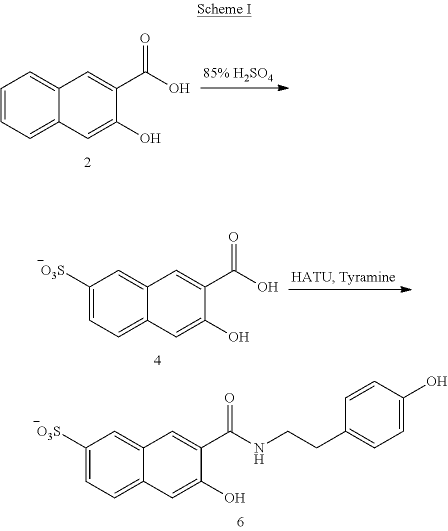

Commercially available carboxy hydroxy naphthalene (2) was sulfonated by treatment with sulfuric acid at elevated temperatures, such as 120 °C. A salt form of 4, such as the sodium salt, can be precipitated from solution as a substantially pure product using NaCl.

Compound 4 can then be coupled to tyramine to form amide 6 using Hexafluorophosphate Azabenzotriazole Tetramethyl Uronium (HATU), a reagent that can be used to form amide bonds. Compound 4 was combined with anhydrous DMF, triethylamine and HATU under an inert atmosphere. Tyramine was added to the reaction mixture, which was scaled and stirred at room temperature overnight. Volatiles were removed, and the residue dissolved in water and filtered. Brine was added to the filtrand, and product (6) precipitated as a fine, slightly yellow solid.

### V. Method of Use

### A. Background

Embodiments of a method for using disclosed compounds in staining protocols, such as immunostaining protocols, for visualizing biological targets also are disclosed. The biological target can be a cell, a tissue, a virus, a gene, an epitope, a target genetic sequence, or a target protein sequence. The present invention can be practiced using specific binding moieties, such as proteins, including antibodies, and may be a multiplexed assay to detect multiple targets in a sample using a plurality of binding agents, such as 2-50 different binding agents (such as antibodies), with each recognizing a specific antigen, to allow detection of a plurality of targets simultaneously. The method may be performed on histologically intact tissue (e.g. formalin-fixed paraffin-embedded tissue (FFPET)) for measuring proteins and other molecules on preserved intact tissues. The intact target tissue may be whole histological sections or, alternatively, of the target may be specific types of cells isolated from histological sections by technologies such as laser capture microdissection, for example.

The present invention can be used to detect proteins in human, animal, and/or plant tissues using antibodies or antibody-like molecules through epitope binding. These detectable proteins include, for example, prognostic and/or predictive factors in cancer, proteins involved in general biological pathways, and/or structural proteins. Certain embodiments of the invention allow screening a sample, such as a patient sample, for a disease marker, such as a tumor-associated marker. In specific embodiments, the binding agent comprises at least one primary antibody, and may include a secondary enzyme-linked antibody, particularly a peroxidase enzyme, such as HRP.

### 1. Processing Tissue Samples

Biological samples may be fixed with a fixative, such as formalin (formaldehyde) and glutaraldehyde, but also may be fixed using other fixation techniques, such as alcohol immersion. The samples may also be embedded in paraffin, and in certain embodiments, the samples are both formalin-fixed and paraffin-embedded. Preparing tissue blocks for IHC and immunostaining procedures is well known to those of ordinary skill in the art. Any intact organ or tissue may be cut into small pieces and incubated in various fixatives until the tissue is “fixed”.

### 2. Deparaffinization of Samples

Deparaffinization substantially or completely removes paraffin from a paraffin-embedded sample. A number of deparaffinization techniques are known, but deparaffinization typically involves washing a sample with an organic solvent, such as benzene, toluene, ethylbenzene, xylenes, and mixtures thereof, to dissolve the paraffin.

### 3. Rehydration

Samples may be rehydrated after deparaffinization, typically using aqueous, lower alcoholic solutions of decreasing concentration. Ethanol is a preferred lower alcohol for rehydration. A sample is mixed with an alcoholic solution and centrifuged.

### 4. Pretreatments

Samples may be pretreated to facilitate making targets available for detection. Pretreatment techniques includes, for example, Heat Induced Epitope Retrieval or Proteolytic Enzyme mediated treatment. Citrate Buffers, Tris, and EDTA base may be used in heat-induced protocols. Proteolytic enzymes include pepsin, proteinase K. and trypsin.

### 5. Antibody Conjugates

Certain embodiments of the present invention concern using antibodies that associate with proteins, polypeptides and peptides. The antibody may be monoclonal. The antibody may be conjugated to a reporter molecule, such as an enzyme. Any antibody of sufficient selectivity, specificity or affinity may be used, as can be evaluated using conventional immunological screening methodologies known to persons of ordinary skill in the art. Antibody conjugates include those linked to an enzyme tag that generates a colored product upon contact with a chromogenic substrate. Any suitable enzyme, such as urease, alkaline phosphatase, and glucose oxidase, may be used, but preferred enzymes for the present invention are peroxidases, such as horseradish peroxidase (HRP), which has a molecular weight 40 kD and is isolated from the root of the horseradish plant. HRP has an iron-containing heme group (hematin) as its active site, which forms a complex with hydrogen peroxide. Noncovalent binding of HRP to an antibody is known to a person of ordinary skill in the art, as described in detail by Stemberger.

Antibodies may be employed for immunodetection of wild-type and/or mutant ligand proteins, polypeptides and/or peptides. Immunodetection methods include enzyme linked immunosorbent assay (ELISA), radioimmunoassay (RIA), immunoradiometric assay, fluoroimmunoassay, chemiluminescent assay, bioluminescent assay, and Western blot. Various useful immunodetection methods are described in the scientific literature, including: Doolittle M H and Ben-Zeev O, 1999; Gulbis B and Galand P, 1993; De Jager R et al., 1993; and Nakamura et al., 1987, each of which is incorporated herein by reference.

Immunobinding generally involves obtaining a sample comprising protein, polypeptide and/or peptide targets. A sample is contacted with a first anti-target antibody under conditions effective to form immunocomplexes, which generally involves adding the antibody composition to the sample and incubating the mixture for a period of time long enough for the antibodies to form immune complexes with antigens present in the sample. Thereafter, the sample-antibody composition, such as a tissue section or Western blot, is washed to remove any non-specifically bound antibody species, to allow detection of only those antibodies involved with primary immune complexes.

### B. Embodiments of the Present Invention

One embodiment of the method comprises providing at least one compound according to Formula I and/or Formula II; and using the compound to detect a target in a sample. The method may comprise, for example, contacting a biological sample comprising a target with a first compound according to Formula I and/or Formula II under conditions selected to covalently associate the first compound to or substantially adjacent the target. The first compound is then contacted with a diazonium compound under conditions that covalently link the first compound to the diazonium compound to form conjugate. The conjugate so formed is then detected using an appropriate technique, such as brightfield or fluorescent microscopy. The first compound may exhibit a first optical signal upon irradiation at an appropriate wavelength of light, and the conjugate may exhibit a second optical signal upon irradiation at an appropriate wavelength of light, wherein the first optical signal and the second optical signal are different.

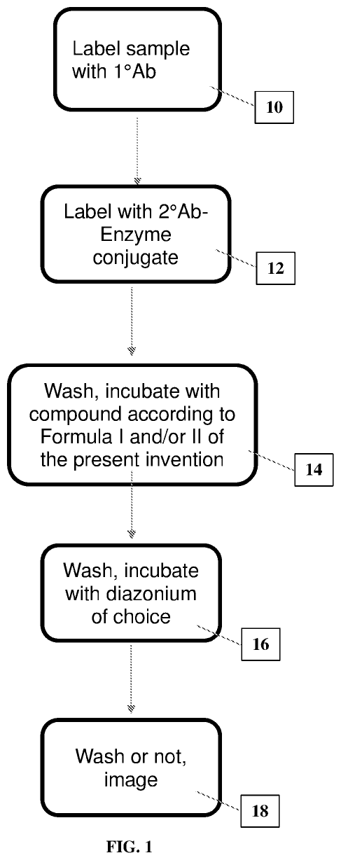

FIGS. 1-3 illustrate various aspects of a disclosed method. With reference to FIG. 1, in a first step 10, a biological sample having a target of interest is contacted with a 1° antibody that associates with an antigenic portion of a target. Exemplary primary antibodies include a Ki67 antibody. The 1° antibody-biological target is then contacted with a 2° antibody to the primary antibody in step 12. Solely by way of example, the 1° antibody may be a mouse antibody, and the 2° antibody a poly HRP-goat anti-mouse (GAM) antibody. As another example, the 1° antibody may be a rabbit antibody, and the secondary antibody a poly-HRP-goat anti-rabbit (GAR). The 2° antibody is conjugated to an enzyme, particularly a peroxidase. Suitable peroxidases include, without limitation, horseradish peroxidase, microperoxidases, and ascorbate peroxidase (APEX).

In step 14, the biological sample associated with the 1° antibody-2° antibody construct optionally may be washed with a buffer solution, such as buffered tween 20. The biological sample is then incubated with a compound according to Formula I and/or Formula II of the present invention in the presence of a peroxide, such as H₂O₂. This process deposits the compound according to Formula I and/or Formula II on or immediately adjacent the desired biological target.

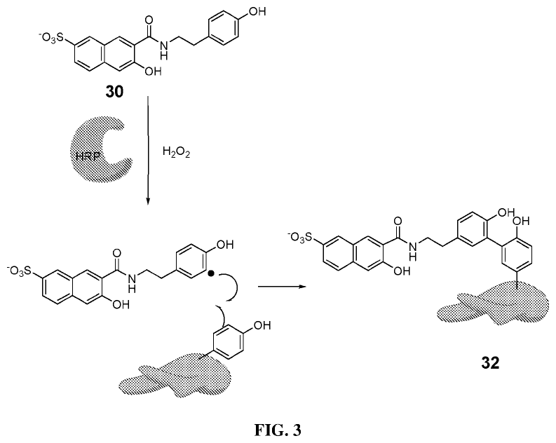

The deposition process is illustrated schematically in FIG. 3. Compound 30 is incubated with the biological sample in the presence of HRP and H₂O₂, which catalyzes the reaction of the exemplary tyramide compound with the target to form tyramide compound-biological sample conjugate 32.

With continuing reference to FIG. 1, the compound-biological sample conjugate is then incubated with a visualization element, such as a colorimetric and/or fluorescent agent, such as a diazonium salt chromogen, in step 16. In step 18, a sample with the visualization element covalently bonded thereto is then optionally washed, and then imaged, such as by using brightfield or fluorescent microscopy.

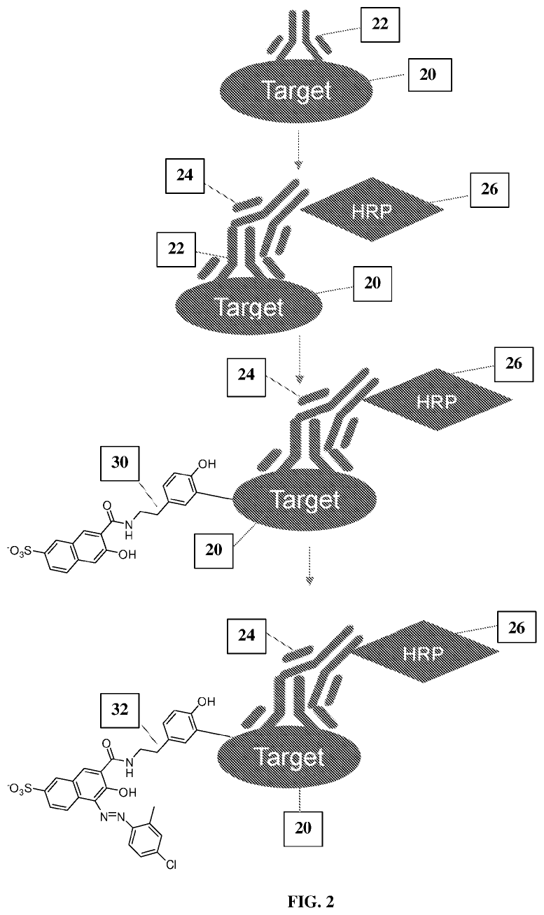

A similar immunostaining procedure is schematically illustrated by FIG. 2. Biological target 20 is associated with 1° antibody 22 to form a biological target-1° antibody conjugate. The biological target-1° antibody conjugate is incubated with a 2° antibody 24 conjugated to a peroxidase, such as the illustrated HRP 26, to form a 1° antibody-2° antibody-HRP conjugate. The 1° antibody-2° HRP conjugate is then incubated with a compound 30 according to the present invention to covalently attach the compound to the target 20. A person of ordinary skill in the art will appreciate that plural such compounds 30 are covalently attached to the target. Subsequent reaction with a diazonium salt forms compounds 32 covalently bound to target 20. This process can be repeated, as desired, using different 1° antibodies and visualization elements to detect, and distinguish, plural different targets in a single sample.

The embodiments illustrated by FIGS. 1-3 involve contacting the target with the first compound in the presence of a peroxidase to covalently couple the first compound to the target. Suitable peroxidases include, without limitation, horseradish peroxidase or ascorbate peroxidase (APEX).

The embodiments illustrated by FIGS. 1-3 also involve coupling a compound according to the present invention with a visualization agent, such as a diazonium salt, typically having a structure such as R—N₂×⁻ where R is an organic group, more typically an organic group having at least one aromatic ring. A person of ordinary skill in the art will appreciate that any diazonium salt can be used in accordance with the present invention. This allows a user to select the color, such as red, violet, blue or green, that the user wants associated with a particular target. Exemplary diazonium compounds include Fast Red TR, Fast Violet LB, and Fast Blue. Conjugates comprising such azo dyes following reaction with an exemplary compound according to the present invention are shown below.

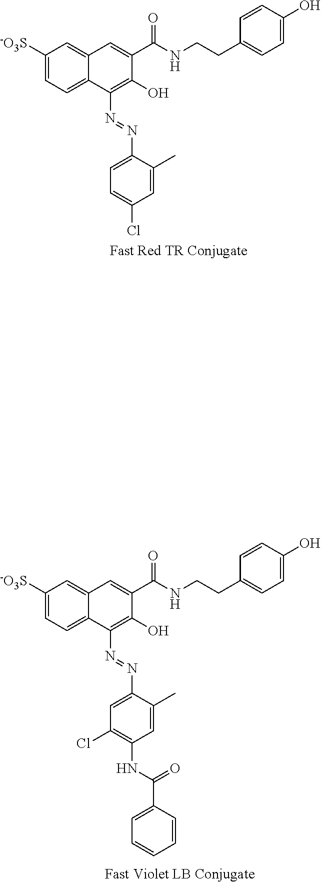

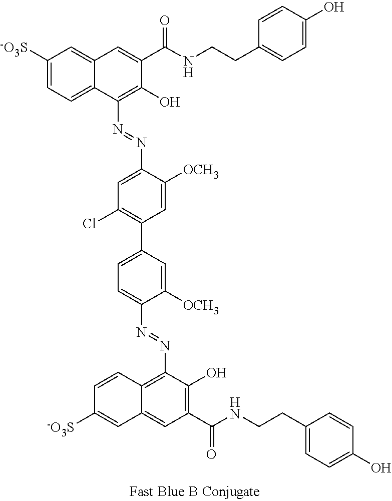

Green may be best produced using an anthracene derivative, as indicated below, where R¹ and R² are, for example,

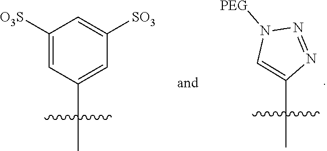

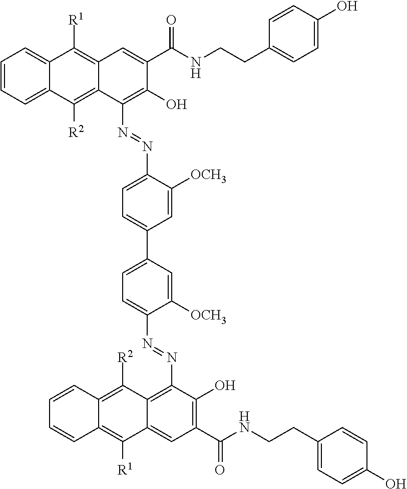

### Fast Blue B Diazonium Salt and Naphthol AS_MX-GR

These conjugates may be detected colorimetrically, such as by using brightfield microscopy. The conjugates also may fluoresce, and hence also may be detected using fluorescent microscopy.

Compounds according to the present invention also can be used in a Western blot protocol. A complete Western-blotting procedure could be carried out and the blots visualized with standard 2^{nd} antibody-HRP conjugates using covalent chromogenic substrates. One embodiment of a Western blot protocol is described it more detail below in Example 5.

### VI. Kits

The present invention also provides kits useful for detecting biological targets. One example of a kit according to the present invention comprises a compound according to Formulas I and/or II, and a diazonium salt. A more particular example of a kit according to the present invention comprises a blocking buffer, such as 3% Bovine Serum Albumin; poly HRP-goat anti-mouse (GAM), poly-HRP-goat anti-rabbit (GAR), and/or HRP-conjugated streptavidin; a compound according to Formula I, II and/or Formula III, such as a 5 mM aqueous solution; a 2 buffer system for tyramide deposition, such as a 200 mM citrate buffer, pH 4, and 7.5% stabilized hydrogen peroxide; a diazonium salt, such as Fast Red TR or Fast Blue BB; and a color development buffer, such as 100 mM Tris, pH 8.5.

### VII. Examples

The following examples are provided to illustrate certain particular features of disclosed embodiments of the present invention. A person of ordinary skill in the art will appreciate that the scope of the invention is not limited to these particular features.

### Example 1—Synthesis of Chromogen Substrates

This example concerns the synthesis of a first chromogen substrate, as indicated below.

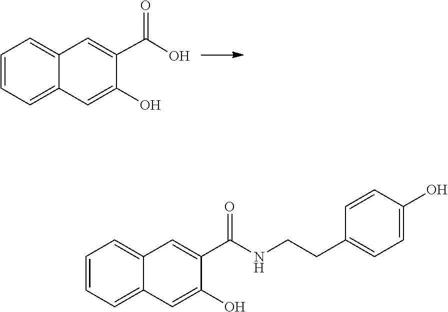 [SMILES: O=C(O)c1cc2ccccc2cc1O ; O=C(NCCc1ccc(O)cc1)c1cc2ccccc2cc1O]

To a round bottom flask at 0 °C was added 3-hydroxynaphthalene-2-carboxylic acid (1.0056 g, 5.3438 mmol), followed by anhydrous tetrahydrofuran (10 mL), then 1-(3-dimethylaminopropyl)-3-ethylcarbodiimide hydrochloride (1.70 mL, 7.8 mmol) via syringe and 1-hydroxybenzotriazole (0.8351 g, 6.057 mmol) and 0.3 mL triethylamine. The reaction mixture was stirred at 0 °C for 1 hour and then warmed to room temperature. Tyramine (5.3438 mmol) was added in one portion and the reaction mixture was stirred overnight under an inert atmosphere. Volatiles were removed, and the product purified using flash chromatography and a eluting system comprising ethyl acetate and hexanes. Pure fractions were combined to provide 3-hydroxy-N-(4-hydroxyphenethyl)-2-naphthamide as a light yellow crystalline solid, 400 mg, 25% of theory. ¹H NMR: (400 MHz, DMSO-d6): δ 12.00, (br s, 1H), 9.21, (s, 1H), 9.07, (t, 1H), 8.48, (s, 1H), 7.84, (d, 1H), 7.73, (d, 1H), 7.50, (t, 1H), 7.35, (t, 1H), 7.26 (s, 1H), 7.07 (d, 2H), 6.68, (d, 2H), 3.52 (dt, 2H), 2.79, (t, 1H).

### Example 2—Synthesis of Chromogen Substrates

This example concerns the synthesis of a second ethoxylated chromogen substrate designed to increase solubility of the substrate in solvents typically used in IHC protocols.

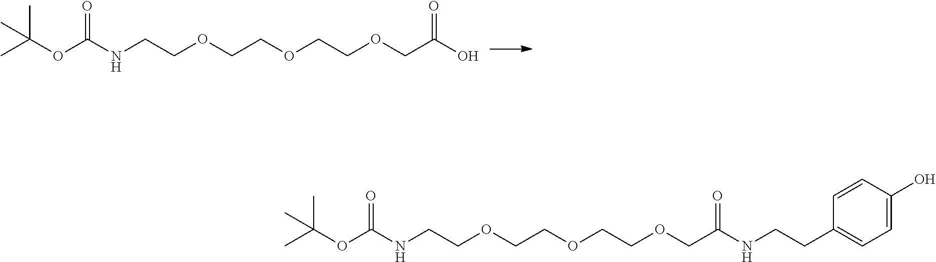 [SMILES: CC(C)(C)OC(=O)NCCOCCOCCOCC(=O)O ; CC(C)(C)OC(=O)NCCOCCOCCOCC(=O)NCCc1ccc(O)cc1]

To a vial was added 2-[2-[2-[2-(tert-butoxycarbonylamino)ethoxy]ethoxy]ethoxy]acetic acid (152.5 mg, 0.4963 mmol), then anhydrous N,N-dimethylformamide (4 mL), then N,N-diisopropylethylamine (0.18 mL, 1.0 mmol), followed by HATU (387 mg, 0.997444 mmol). The reaction mixture was stirred for 15 minutes. 4-(2-aminoethyl)phenol (72.1 mg, 0.526 mmol) was then added to the reaction mixture in one portion. The reaction mixture was stirred overnight at room temperature under an inert atmosphere. Volatiles were removed, and the reaction mixture was purified using flash chromatography and H₂O/MeCN. The pure fractions were combined to yield tert-butyl (1-(4-hydroxyphenyl)-4-oxo-6,9,12-trioxa-3-azatetradecan-14-yl)carbamate as a yellow oil, 191.2 mg, 90% of theory. ¹H NMR: (400 MHz, DMSO-d6): δ 7.05, (d, 2H), 6.72, (d, 2H), 3.97, (s, 2H) 3.7-3.0, (m, 16H) 1.38, (s, 9H).

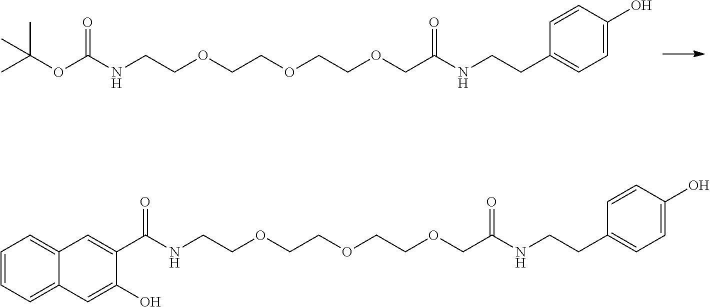 [SMILES: CC(C)(C)OC(=O)NCCOCCOCCOCC(=O)NCCc1ccc(O)cc1 ; O=C(COCCOCCOCCNC(=O)c1cc2ccccc2cc1O)NCCc1ccc(O)cc1]

In a vial containing 100 mg of tert-butyl (1-(4-hydroxyphenyl)-4-oxo-6,9,12-trioxa-3-azatetradecan-14-yl)carbamate was added 5 mL dichloromethane, followed by 1 mL of trifluoroacetic acid. The mixture was stirred stir for 2 hours, until consumption of starting material was confirmed by thin layer chromatography (TLC). Volatiles were removed, and then anhydrous N,N-dimethylformamide (1 mL) was added to the reaction mixture.

In a separate vial, 3-hydroxynaphthalene-2-carboxylic acid (60 mg, 0.319 mmol), anhydrous N,N-dimethylformamide (5 mL), N,N-diisopropylethylamine (0.1 mL, 0.6 mmol), and lastly HATU (241 mg, 0.621 mmol) were combined. The reaction mixture was allowed to stir for 15 minutes. A DMF solution of 2-(2-(2-(2-aminoethoxy)ethoxy)ethoxy)-N-(4-hydroxyphenethyl)acetamide was added, and the reaction mixture was stirred overnight at room temperature under an inert atmosphere. Volatiles were removed, and the reaction mixture was purified using flash chromatography with H₂O/MeCN. The pure fractions were combined to yield 3-hydroxy-N-(1-(4-hydroxyphenyl)-4-oxo-6,9,12-trioxa-3-azatetradecan-14-yl)-2-naphthamide as a tacky light-yellow solid, 80.1 mg, 53% of theory. ¹H NMR: (400 MHz, CD3OD): δ 8.69, (s, 1H), 8.07, (d, 1H), 7.97, (d, 1H), 7.69, (t, 1H), 7.59, (t, 1H), 7.00, (d, 2H), 6.70, (d, 2H), 4.31. (t, 2H), 3.89-3.75 (m, 4H), 3.70-3.25. (m, 12H), 2.68, (t, 2H).

### Example 3—Synthesis of Chromogen Substrates

This example concerns the synthesis of a third sulfonated chromogen substrate designed to increase solubility of the substrate in solvents typically used in IHC protocols.

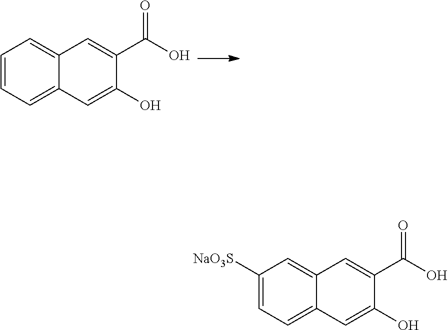

To a round bottom flask was added 42 mL 85% H₂SO₄. The flask was heated to 120 °C with stirring, and then 17.4 g 3-hydroxy-2-naphthoic acid was added and the resulting reaction was stirred for 30 minutes. The reaction mixture was then removed from heat and allowed to cool to room temperature. The reaction mixture was diluted with 275 mL deionized water and heated to 60 °C, and then 16 grams NaCl were added. The solution was stirred for 10 minutes, and then removed from heat and allowed to cool to room temperature with stirring. The reaction mixture was stirred for 1 hour at room temperature. The product, sodium 7-carboxy-6-hydroxynaphthalene-2-sulfonate, precipitated as a fine white solid and was collected via vacuum filtration and dried under high vacuum overnight. Sodium 7-carboxy-6-hydroxynaphthalene-2-sulfonate was isolated as a free-flowing white solid, 26.3 grams, 97% of theory. ¹H NMR: (400 MHz, DMSO-d6): δ 11.01, (br s, 1H), 8.56 (s, 1H), 8.16 (s, 1H), 7.74 (m, 2H), 7.33 (s, 1H).

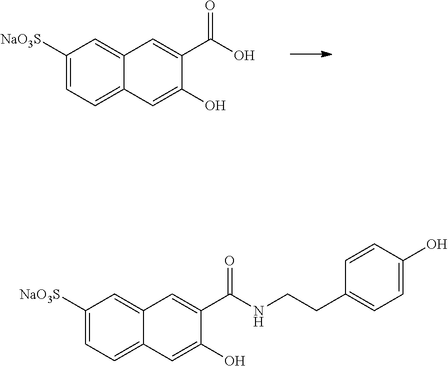

To a round bottom flask was added 26 grams of sodium 7-carboxy-6-hydroxynaphthalene-2-sulfonate, followed by 100 mL anhydrous DMF and 27 mL of triethylamine under an inert atmosphere. After 10 minutes, HATU (35 g) was added, and the reaction mixture stirred for an additional 10 minutes. 12.61 grams of tyramine were added to the reaction mixture, which was sealed and stirred at room temperature overnight. Volatiles were removed, and the residue dissolved in 300 mL water and filtered. 60 mL brine were added to the filtrand, and the resulting solution was allowed to stand for 30 minutes. Product precipitated as a fine, slightly yellow solid, which was collected via vacuum filtration and dried via high vacuum. Sodium 6-hydroxy-7-((4-hydroxyphenethyl)carbamoyl)naphthalene-2-sulfonate was isolated as a free-flowing, light yellow solid, 13 g, 35% of theory. ¹H NMR: (400 MHz, DMSO-d6): δ 12.16 (br s, 1H), 9.22, (s, 1H), 9.08 (t, 1H), 8.51, (s, 1H), 8.07, (s, 1H), 7.69 (m, 2H), 7.26 (s, 1H), 7.77 (d, 2H), 6.70 (d, 2H), 3.53 (dt, 2H), 2.80 (t, 2H).

### Example 4—Tissue Processing and Brightfield Imaging

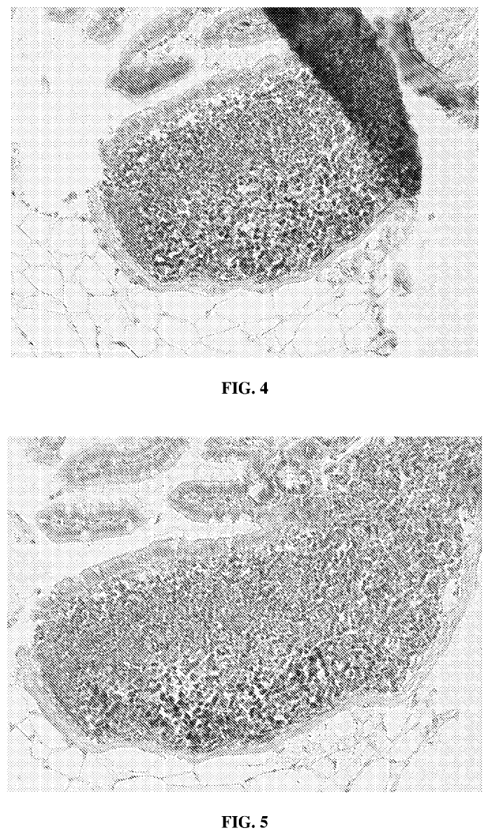

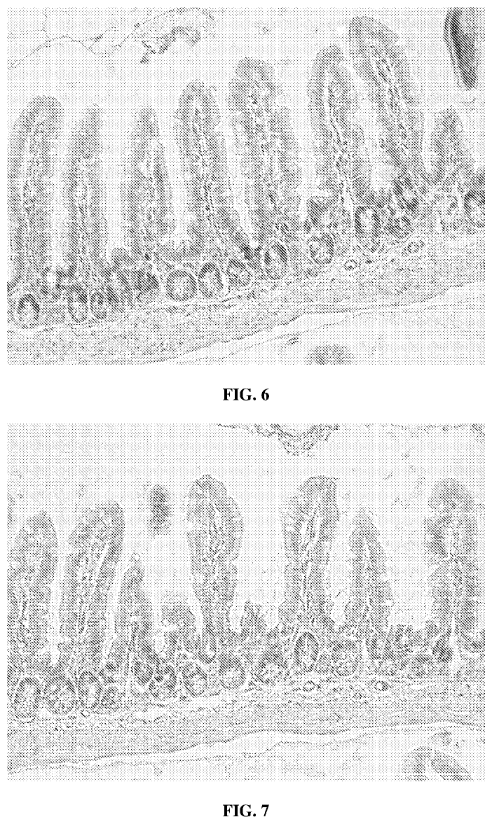

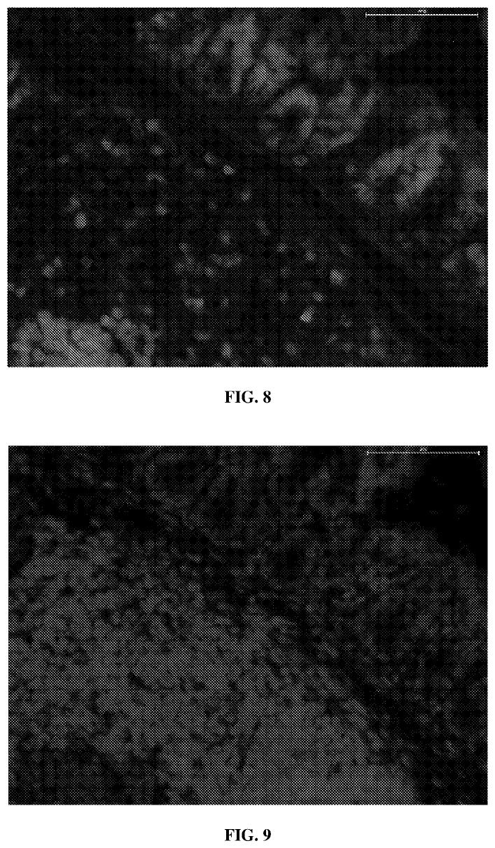

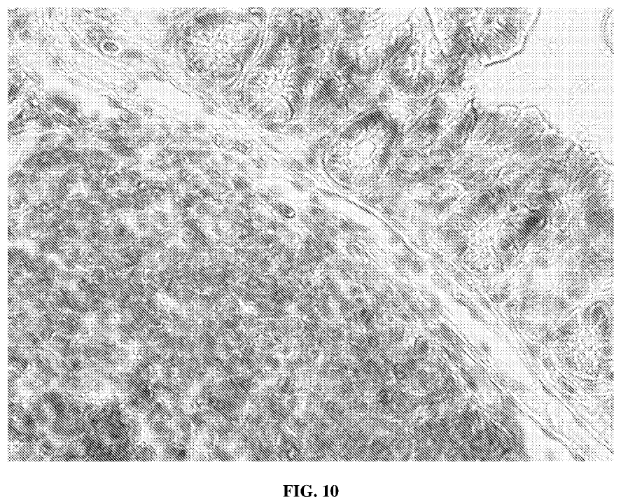

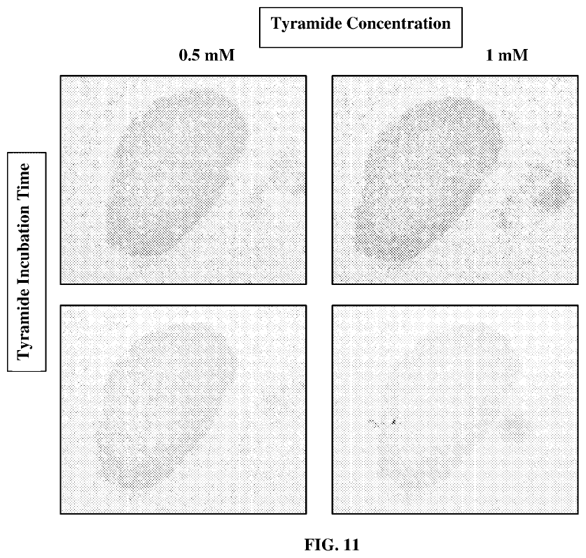

Fixed tissue was processed via deparaffinization and heat induced epitope retrieval (HIER). The tissue was then permeabilized with buffered 0.1% triton for 30 minutes and blocked with the appropriate normal serum, based on species of primary antibody, for 1 hour at room temperature. The primary antibody was diluted to desired concentration and added to tissue. Tissue was then incubated for desired time at room temperature in a humidified chamber. Slides are then washed in buffered 0.05% tween 20. A secondary antibody conjugate (SuperBoost™ Poly HRP with appropriate species to match primary) was diluted to desired concentration and added to tissue, which was incubated for 1 hour at ambient temperature in a humidified chamber. The slides were washed in buffered 0.05% tween 20. Sodium 6-hydroxy-7-((4-hydroxyphenethyl)carbamoyl)naphthalene-2-sulfonate was diluted in citrate buffer with 3.75% H₂O₂ and incubated for desired time at ambient temperature in a humidified chamber. Slides were then washed in buffered 0.05% tween 20. Color was then developed by selection of appropriate reagents (red, blue, purple, or DAB control) for a time period selected to obtain desired intensity. Color development was stopped using SHAM when desired staining intensity was reached. The sample was co-stained with Gill's hematoxylin and/or DAPI as desired, and the slide imaged using a brightfield microscope. FIGS. 4-10 provide images of samples stained according to this example.

### Example 5—Western Blot

This example describes using a covalent chromogenic substrate for protein visualization in a Western blotting application A complete Western-blotting procedure could be carried out and the blots visualized with standard 2^{nd} antibody-HRP conjugates using the covalent chromogenic substrates

The protein extract of interest is diluted to the appropriate desired concentrations in Laemmli buffer (20% Glycerol, 2.1% SDS, 0.125 M Tris, pH 6.8, 0.73 M 2-mercaptoethanol, bromophenol blue), then the solution is heated in a 95-100 °C water bath for five minutes, cooled to room temperature and loaded onto SDS-PAGE gels using acrylamide percentage appropriate for the protein of interest plus stacking (8% acrylamide with bromophenol blue). A pre-stained protein ladder (10-170 kDa) can also be added as a molecular weight reference. Electrophoresis is performed at 200 V until the dye front runs off the gel, which typically is about one. The proteins are then transferred from the eel to the PVDF membrane at 100 V for one hour at 6 °C. The PVDF membrane is washed twice with TTBS (5 minutes each), and then the membrane is blocked with milk powder in TTBS, followed by incubation with primary antibody selected tor the protein target of interest. The PVDF is then rewashed and incubated with secondary antibody-HARP conjugate. Covalent reaction of the substrate is performed by the incubation of the covalent chromogenic substrate (compound 5) in HRP buffer containing H₂O₂ followed by a TTBS wash then incubation of the diazonium salt of the desired color. The reaction with die diazonium salt w as rapid.

Proteins could be, detected with a straightforward procedure that does not require any additional equipment or time compared to a procedure with traditional colorimetric probes using NBT/BCIP visualization of protein target. In addition to colorimetric signal, some of the diazonium salts are also fluorescent. This method can be easily extended for multiplexed detection and be widely applied for protein analysis.

### Example 6

This example evaluates the results obtained using a Formula I

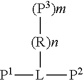

compound according to the present invention, namely

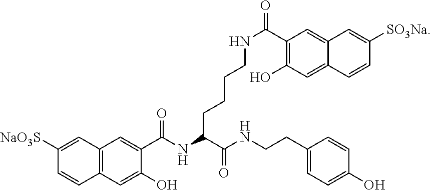

This compound was evaluated to determine the effects of chromogen concentrations and incubation times on staining results. This example and FIG. 11 establish that chromogens according to the present invention provide highly suitable staining results for typical staining protocols. Higher chromogen concentrations provide more intense staining results, as can be seen by comparing images of FIG. 11 that were produced using 0.5 mM and 1 mM concentrations. However, increased incubation times also can result in over development and less intense staining results, as can be seen by comparing images produced using a 10-minute incubation time to a 30-minute incubation times.

### Example 7

This example compares results obtained for Ki67 tonsil tissue staining that used: (1) a first formula II P¹-L-P² compound according to the present invention, namely

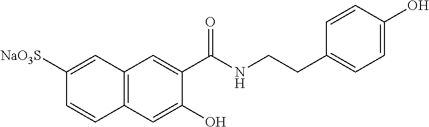

in a 1 mM staining solution that provided a total compound concentration of 1 mM; a second Formula I

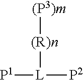

compound according to the present invention, namely

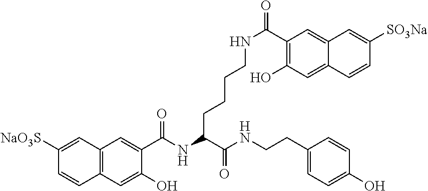

in an 0.5 mM staining solution that provided a total compound concentration of 1 mM; and the well-known diaminobenzidine (DAB),

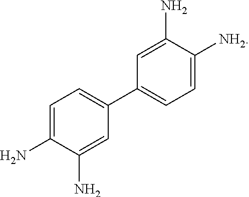 [SMILES: Nc1ccc(-c2ccc(N)c(N)c2)cc1N]

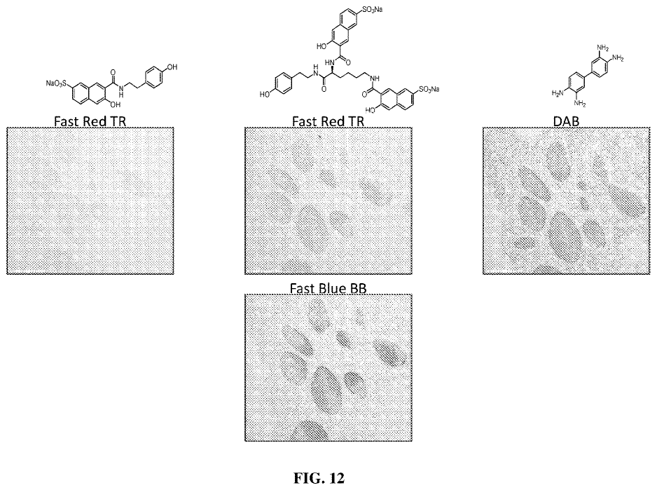

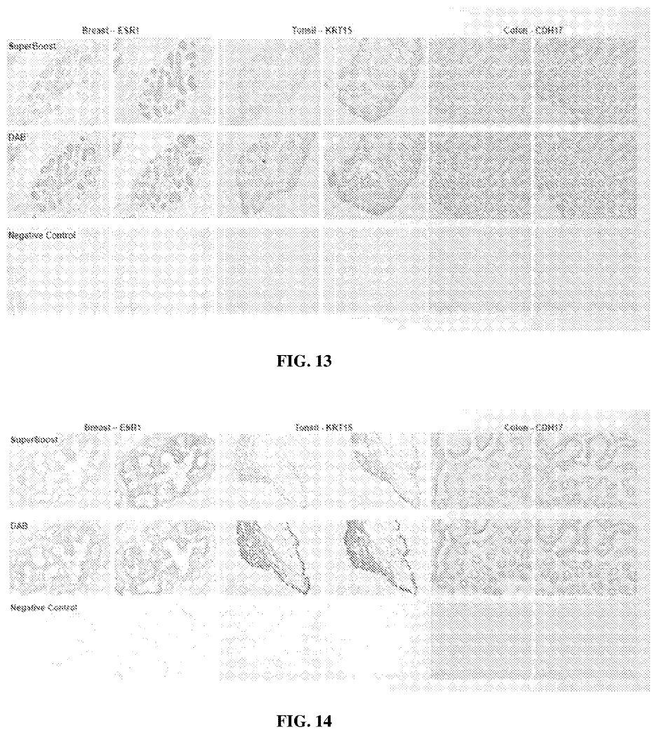

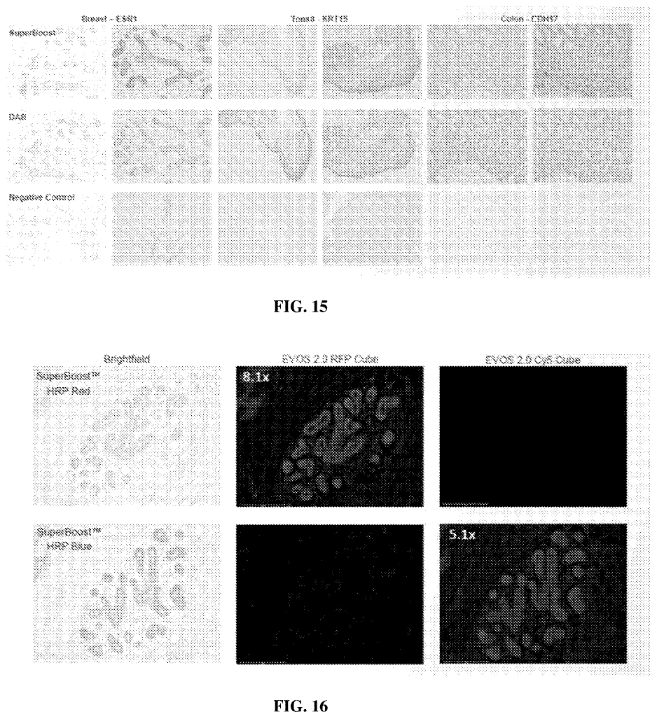

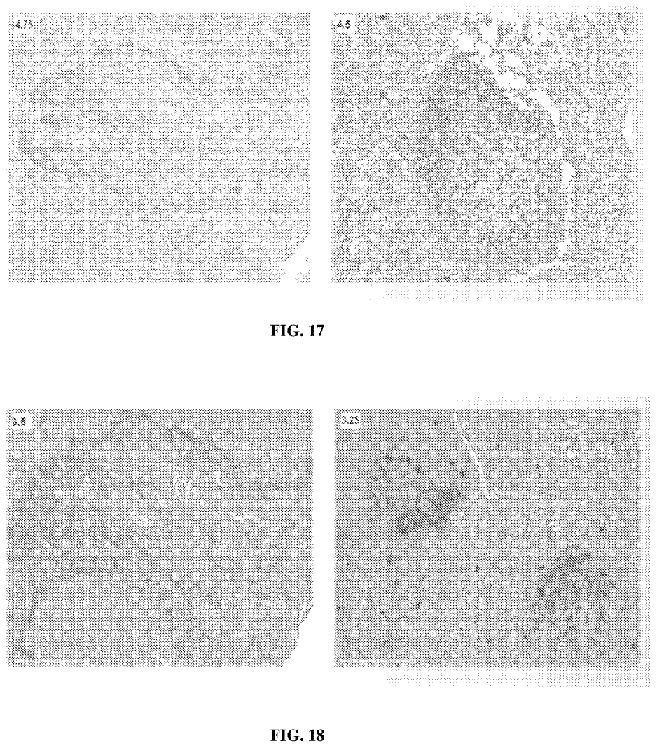

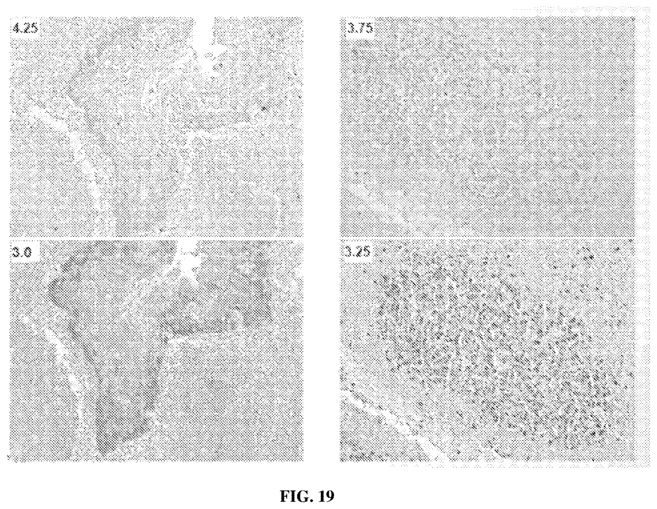

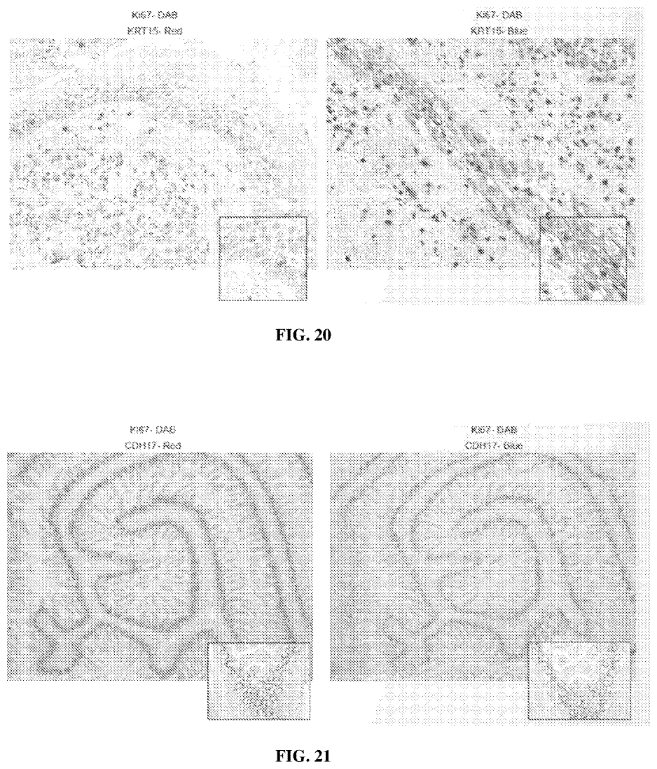

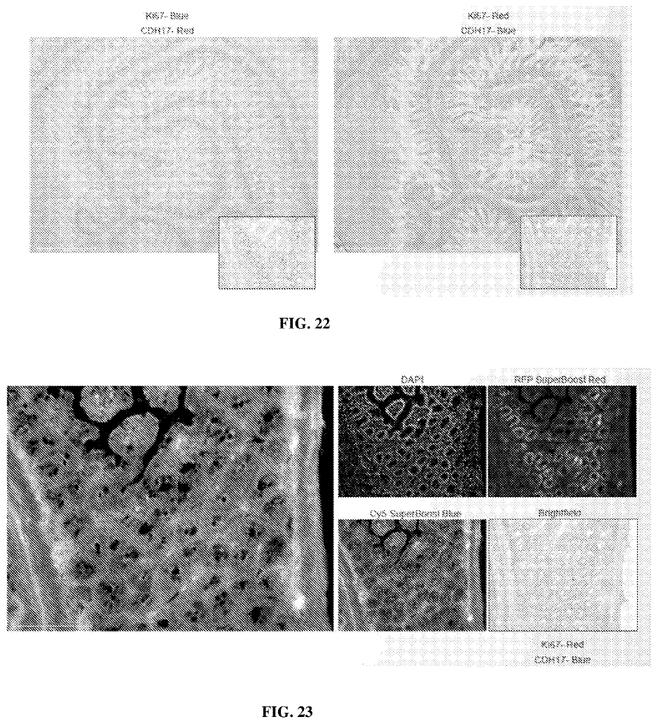

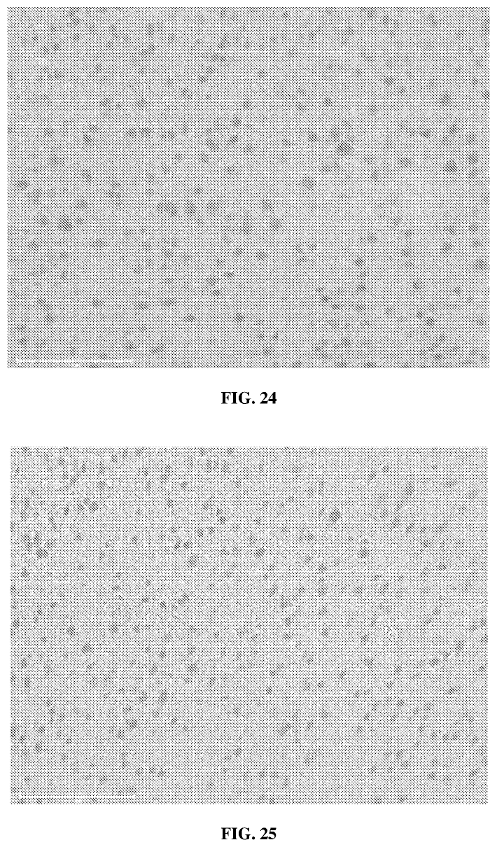

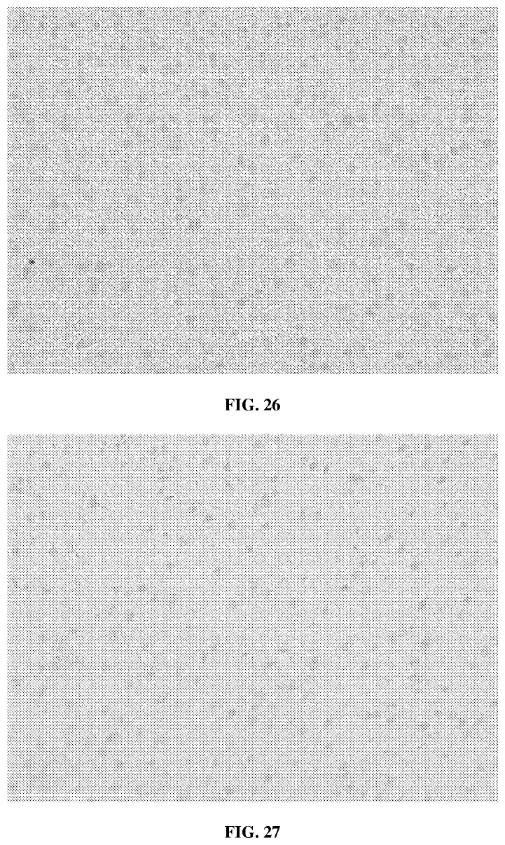

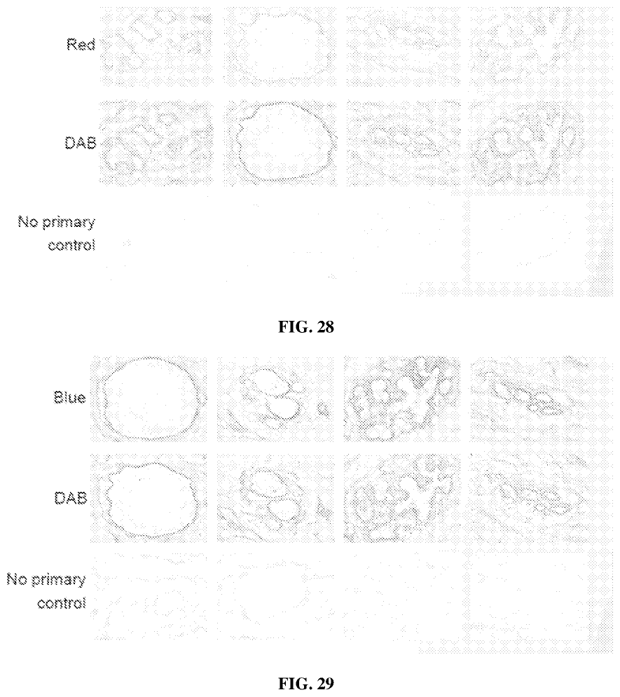

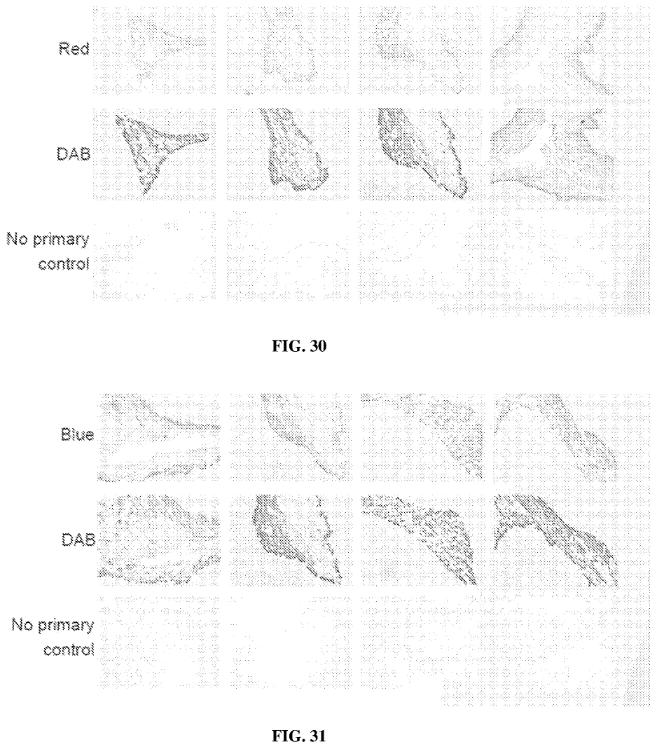

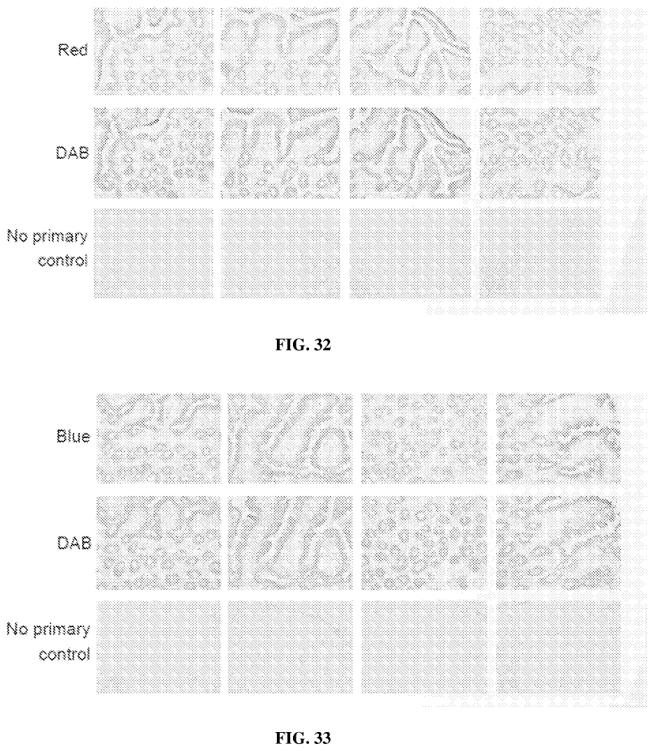

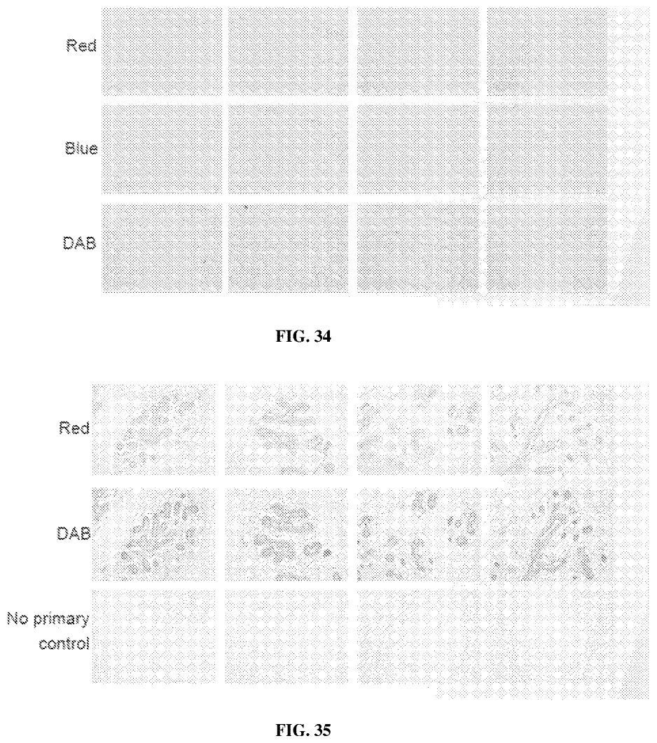

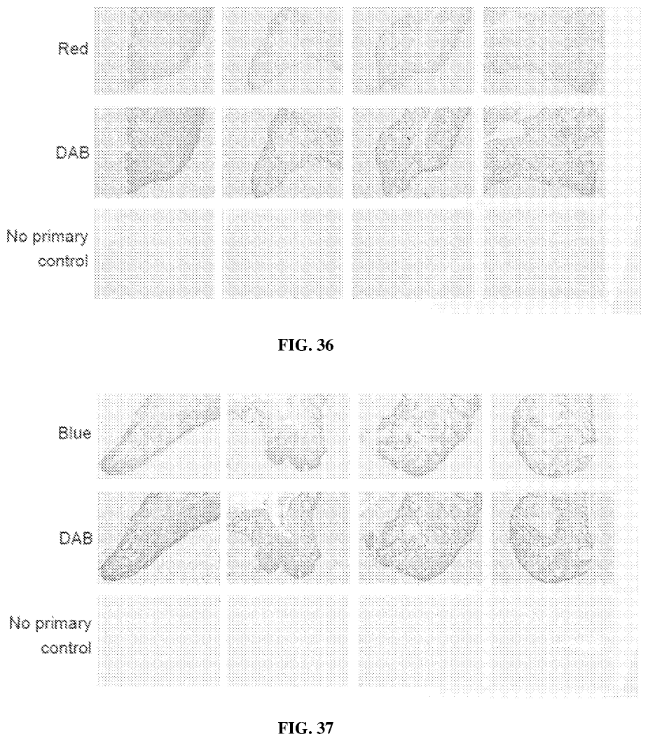

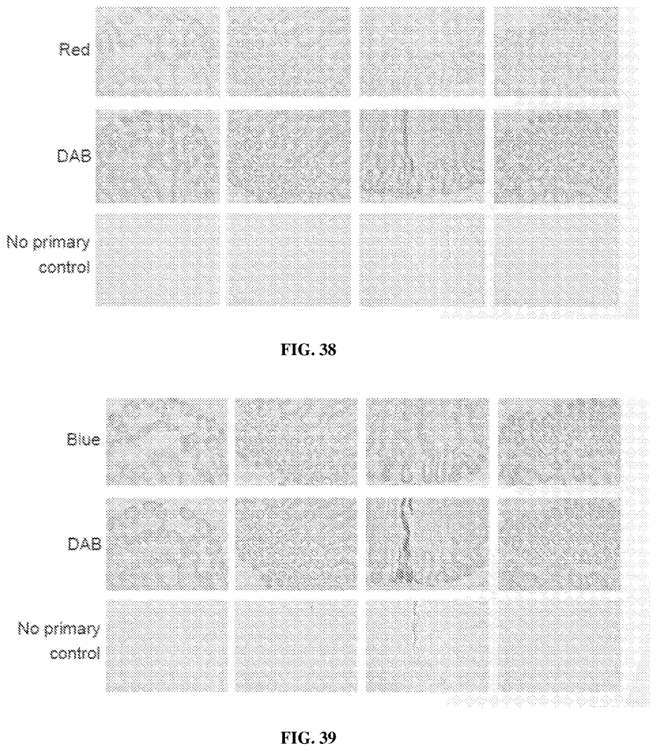

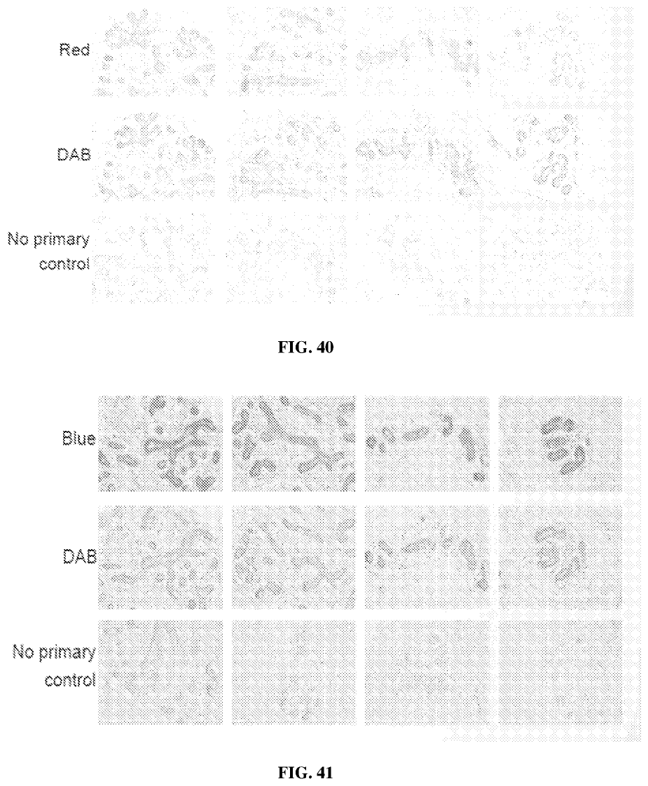

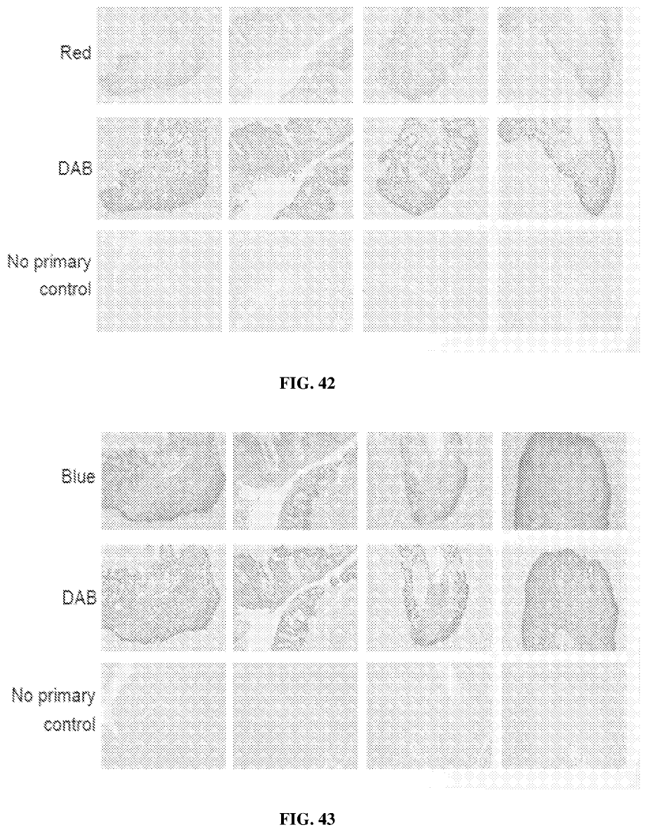

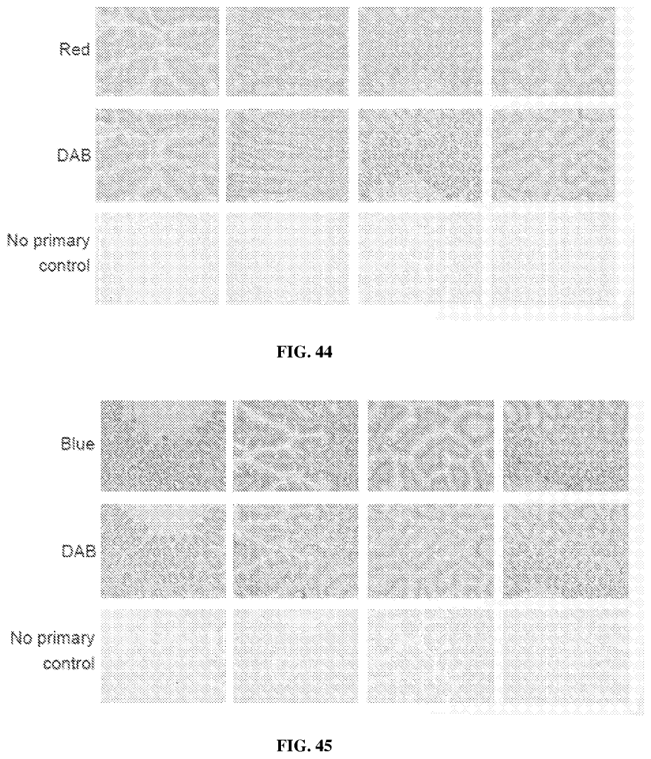

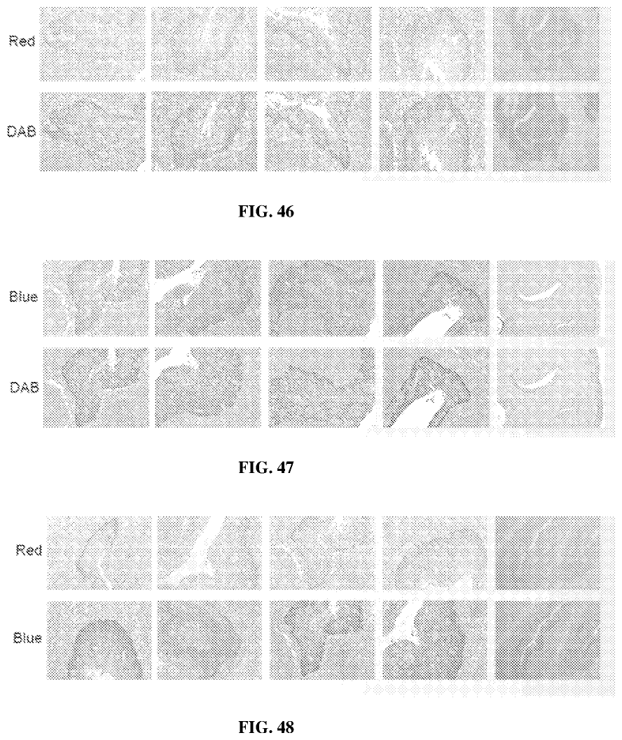

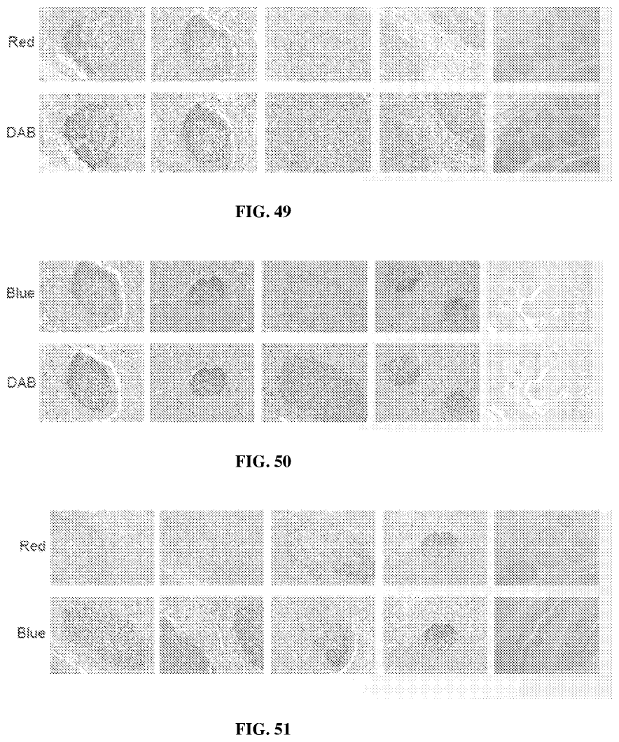

While all 3 tests produced suitable staining images (See FIG. 12), the second compound appears to provide a more intense Fast Red stain than the first compound, and can be used in a wider array of staining protocols than can DAB. The second compound further proved to be a viable and robust histochemical stain, detectable and multiplexable via brightfield and fluorescence imaging in both blue and red colors, as demonstrated in FIGS. 13-51, compatible with a variety of tissue types, counterstains, and mounting media.

### Example 8

This example concerns synthesis of di-tert-butyl (6-((4-hydroxyphenethyl)amino)-6-oxohexane-1,5-diyl)(S)-dicarbamate according to the following scheme.

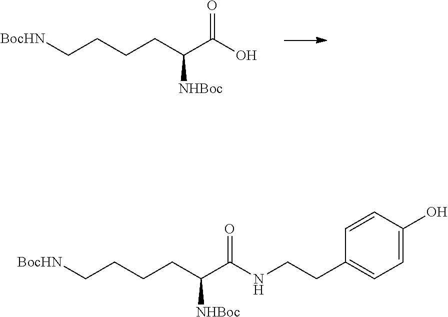 [SMILES: CC(C)(C)OC(=O)NCCCC[C@H](NC(=O)OC(C)(C)C)C(=O)O ; CC(C)(C)OC(=O)NCCCC[C@H](NC(=O)OC(C)(C)C)C(=O)NCCc1ccc(O)cc1]

To a solution of N²,N⁶-bis(tert-butoxycarbonyl)-L-lysine (1 g, 2.9 mmol) in anhydrous DMF (30 mL) was added triethylamine (0.5 mL, 3.6 mmol, 1.2 eq), followed by HATU (1.323 g, 3.5 mmol, 1.2 eq), and tyramine (477 mg, 3.5 mmol, 1.2 eq) and the solution was stirred for 18 hours. Volatiles were removed via rotovap, then residue was dissolved in EtOAc (100 mL). The solution was washed with 0.5 M HCl (2×50 mL), DI water (1×50 mL), sat. NaHCO₃, and brine (1×50 mL). Organic layer was dried over Na₂SO₄, filtered, and then volatiles were removed via rotovap. Purified via flash chromatography, mobile phase MeOH/CHCl₃ to yield 951 mg of di-tert-butyl (6-((4-hydroxyphenethyl)amino)-6-oxohexane-1,5-diyl)(S)-dicarbamate as a brownish oil, 70% of theory. ¹H NMR, DMSO-d6: 7.01 (d, 2H), 6.82 (d, 2H), 6.15 (br s, 1H), 5.22 (br s, 1H), 4.75 (br s, 1H), 4.00 (br s, 1H), 3.60 (br s, 1H), 3.40 (br s, 1H), 3.01 (m, 2H), 2.71 (m, 2H), 1.62 (br s, 2H), 1.51-1.25 (m, 20H), 1.12 (br s, 2H).

### Example 9

This example concerns the synthesis of (S)-6-((4-hydroxyphenethyl)amino)-6-oxohexane-1,5-diaminium 2,2,2-trifluoroacetate according to the following scheme.

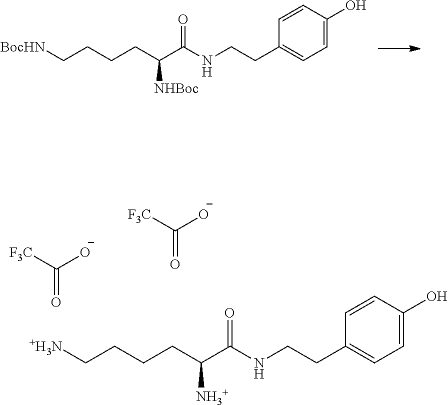 [SMILES: CC(C)(C)OC(=O)NCCCC[C@H](NC(=O)OC(C)(C)C)C(=O)NCCc1ccc(O)cc1 ; O=C([O-])C(F)(F)F ; [NH3+]CCCC[C@H]([NH3+])C(=O)NCCc1ccc(O)cc1 ; O=C([O-])C(F)(F)F]

To a solution of di-tert-butyl (6-((4-hydroxyphenethyl)amino)-6-oxohexane-1,5-diyl)(S)-dicarbamate (950 mg, 2.0 mmol) in dichloromethane (20 mL) was added trifluoroacetic acid (5 mL, 65.4 mmol). The solution was stirred for 3 hours, after which volatiles were removed via rotovap. Dried under highvac for 18 hours to yield (S)-6-((4-hydroxyphenethyl)amino)-6-oxohexane-1,5-diaminium 2,2,2-trifluoroacetate as a tacky brown solid, quantitative conversion.

### Example 10

This example concerns the synthesis of triethylammonium (S)-7,7′-(((6-((4-hydroxyphenethyl)amino)-6-oxohexane-1,5-diyl)bis(azanediyl))bis(carbonyl))bis(6-hydroxynaphthalene-2-sulfonate) according to the following scheme.

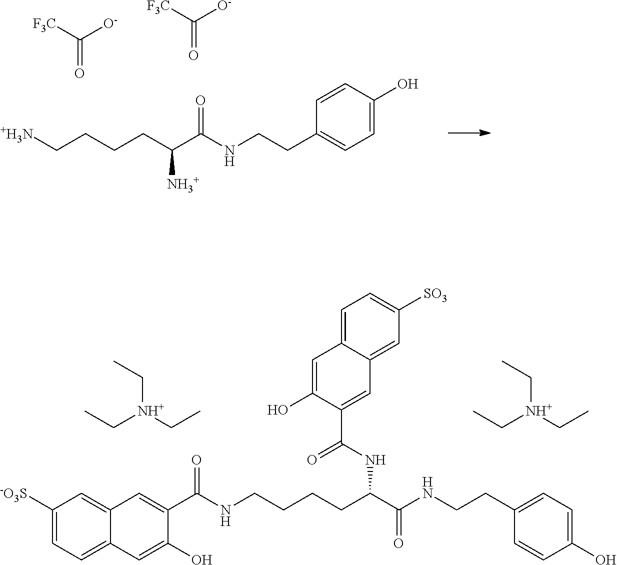

To a solution of sodium 7-carboxy-6-hydroxynaphthalene-2-sulfonate (1.161 g, 4.0 mmol, 2 eq) in anhydrous DMF (30 mL) was added HATU (1.597 g, 4.2 mmol, 2.1 eq) and triethylamine (0.8 mL, 5.8 mmol, 2.9 eq). After stirring for 1 hour, (S)-6-((4-hydroxyphenethyl)amino)-6-oxohexane-1,5-diaminium 2,2,2-trifluoroacetate (987 mg, 2 mmol) was added as a 200 mM solution in anhydrous DMF, and an additional aliquot of TEA is added (0.8 mL, 5.8 mmol, 2.9 eq). The solution was stirred for 18 hours, then volatiles were removed via rotovap. Purified via reverse phase flash chromatography, mobile phase MeOH/water, to yield 1.065 g of triethylammonium (S)-7,7′-(((6-((4-hydroxyphenethyl)amino)-6-oxohexane-1,5-diyl)bis(azanediyl))-bis(carbonyl))bis(6-hydroxynaphthalene-2-sulfonate) as a light yellow solid, 55% of theory. ¹H NMR, DMSO-d6: 9.13 (s, 1H), 9.03 (t, 1H), 8.64 (s, 1H), 8.52 (s, 1H), 8.18 (t, 1H), 8.07 (d, 2H), 7.75-7.6 (m, 4H), 7.26 (d, 2H), 6.97 (d, 2H), 6.64 (d, 2H), 4.53 (q, 1H), 3.15-3.4 (m, 4H), 2.6 (t, 2H), 1.9-1.7 (m, 2H), 1.63 (m, 2H), 1.41 (n, 2H).

In view of the many possible embodiments to which the principles of the disclosed invention may be applied, it should be recognized that the illustrated embodiments are only preferred examples of the invention and should not be taken as limiting the scope of the invention. In general, in the following clauses and claims, the terms used should not be construed to limit the clauses and claims to the specific embodiments disclosed in the specification and the claims, but should be construed to include all possible embodiments along with the full scope of equivalents to which such clauses and claims are entitled. Accordingly, the clauses and claims are not limited by the disclosure. We therefore claim as our invention all that comes within the scope and spirit of these claims. In addition, embodiments may be in accordance with the following numbered clauses.

1. A compound, satisfying Formula I

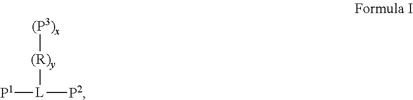

where:
L is a linker having from 1 to at least 50 atoms;
P¹, P² and P³ are aromatic rings or aromatic ring systems;
R is a bond, or is independently an L group; and
x and y independently are 0 to 5.

2. The compound according to clause 1 wherein P¹ and P³ ring systems are the same or different and each may comprise at least two fused rings, wherein at least one of the fused rings comprises a structure represented as

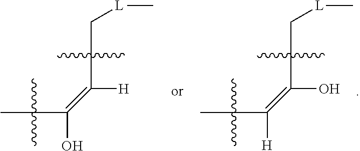

3. The compound according to clauses 1 or 2, wherein P² is a phenyl ring substituted with at least one —OH group.

4. The compound according to any of clauses 1-3 wherein:
L is a linker having from 2 to 20 atoms in a chain;
R is a bond, or is independently an L group; and
x and y independently are 0 or 1.

5. The compound according to clause 1, having a structure selected from

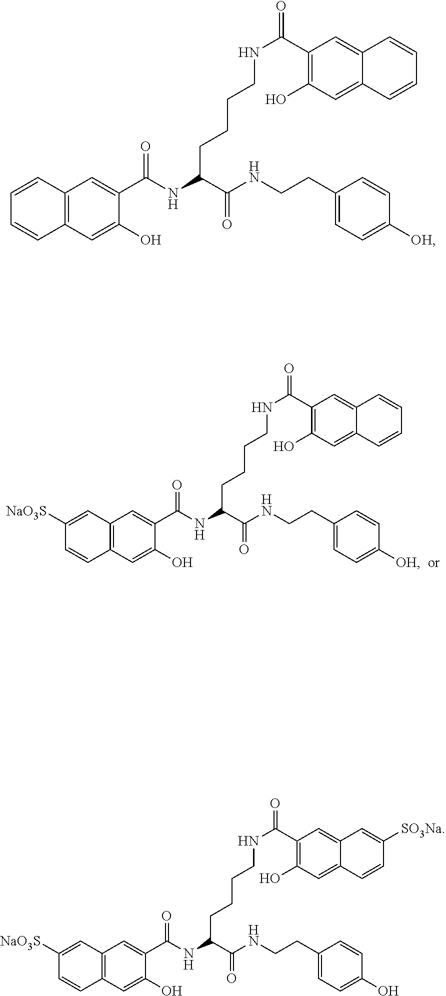

6. The compound according to clause 1, satisfying Formula II

P¹-L-P², Formula II

where:
P¹ is a first aromatic ring system comprising at least two fused rings, wherein at least one of the fused rings comprises a structure represented as

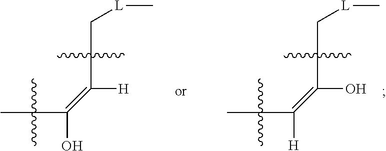

P² is a second aromatic ring system substituted with at least one —OH group; and
L is a linker.

7. The compound according to clause 6, wherein P¹ has from 2 to 4 rings in a fused-ring system.

8. The compound according to any of clauses 1-7, wherein P¹ and/or P³ is substituted with at least one aqueous solubility moiety selected to increase the aqueous solubility of the compound.

9. The compound according to any of clauses 1-8, wherein P¹ is selected from:

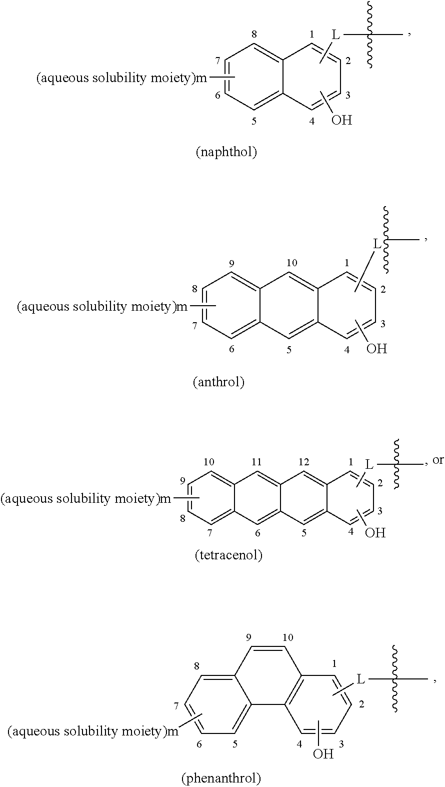

where m is from 1 to 5.

10. The compound according to clauses 8 or 9, wherein the aqueous solubility moiety is selected from SO₃⁻, —SO₃H, —CO₂⁻, —CO₂H, —HPO₃⁻², H₂PO₃⁻¹, H₃PO₃, PEG,

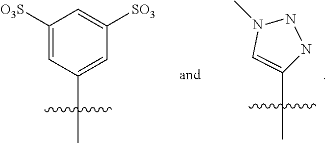

11. The compound according to any of clauses 1-10 where a hydroxyl group on P¹ is ortho or meta to L.

12. The compound according to clause 9, wherein:
P¹ is naphthol, L is coupled to position 2 or 3, at least one hydroxyl group is at positions 3 and/or 4, and the at least one moiety selected to increase the aqueous solubility of the compound is coupled to at least one of the 5-8 positions;
P¹ is anthrol, L is coupled to position 2 or 3, at least one hydroxyl group is at positions 3 and/or 4, and the at least one moiety selected to increase the aqueous solubility of the compound is at coupled to at least one of the 6-9 positions;
P¹ is chrysenol, L is coupled to position 2 or 3, at least one hydroxyl group is at positions 3 and/or 4, and the at least one moiety selected to increase the aqueous solubility of the compound is at coupled to at least one of the 7-10 positions; or
P¹ is phenanthrol, L is coupled to position 2 or 3, at least one hydroxyl group is at positions 3 and/or 4, and the at least one moiety selected to increase the aqueous solubility of the compound is at coupled to at least one of the 5-8 positions.

13. The compound according to clause 1 where P¹ and/or P³ is a heteroaryl fused ring system.

14. The compound according to clause 13 where the heteroaryl fused ring system is electron rich.

15. The compound according to clauses 13 or 14 wherein the heteroaryl fused ring system is selected from fused furan, fused thiophene, and fused pyrrole ring systems.

16. The compound according to any of clauses 13-15, where P¹ and/or P³ is selected from

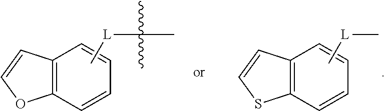

17. The compound according to any of clauses 13-16, wherein P¹ and/or P³ is substituted with at least one moiety selected to increase the aqueous solubility of the compound.

18. The compound according to any of clauses 1-17, wherein P² is an aromatic hydrocarbon.

19. The compound according to any of clauses 1 to 18, wherein P² comprises a phenol.

20. The compound according to clause 19 wherein the phenol has a hydroxyl group that is para to L.

21. The compound according to any of clauses 1-20, wherein L is selected from C₁-C₅₀ aliphatic, C₁-C₅₀ alkyl, C₁-C₅₀ alkyl amide, C₁-C₅₀ alkyl ester, C₁-C₅₀ ketone, a C₂-C₅₀ polyol, C₂-C₅₀ polyalkyl ethers, and C₂-C₅₀ aliphatic amines.

22. The compound according to any of clauses 1-21, wherein:
P¹ is selected from

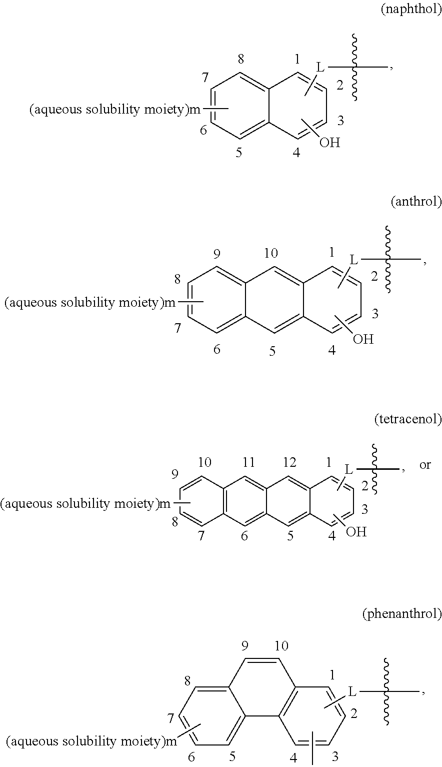

where n is from 1 to 3;
P² comprises a phenol;
the at least one moiety selected to increase the aqueous solubility of the compound is selected from —SO₃⁻, —SO₃H, —CO₂⁻, —CO₂H, —HPO₃⁻², H₂PO₃⁻¹, H₃PO₃, PEG,

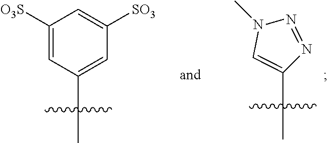

and
L is selected from a covalent bond, C₁-C₁₀ alkyl, a C₁-C₁₀ alkyl amide, a C₁-C₁₀ ketone, or a C₁-C₁₀ glycol.

23. The compound according to clause 22, where:
P¹ is naphthol;
m is 1; and
the at least one moiety selected to increase the aqueous solubility is —SO₃⁻.

24. The compound according to clauses 21-23, where L is

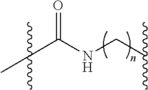

and n is from 1 to 5.

25. The compound according to any of clauses 21-23, where m is 1 and n is 2.

26. The compound according to clause 1, where:
P¹ is naphthol;
P² is phenol;
the at least one moiety selected to increase the aqueous solubility is —SO₃⁻; and
L is

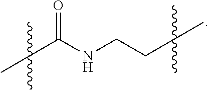 [SMILES: *CCNC(*)=O]

27. A compound, having a structure

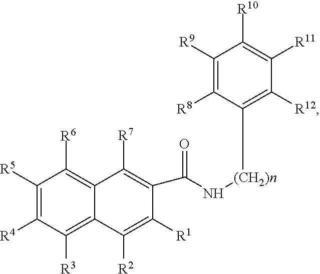

where:
R¹ and R² are independently selected from H and OH;
R³-R⁸ are independently selected from H, —F, —Cl, —Br, —I, —OH, —OR⁹ where R⁹ is C₁-C₆ alkyl, —SO₃⁻, —SO₃H, —CO₂⁻, —CO₂H, —HPO₃⁻², H₂PO₃⁻¹, H₃PO₃, PEG,

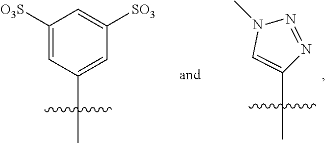

where at least one of R¹, R², R⁷ and at least one of R⁸-R¹² is hydroxyl; or
R₃ and R₄ taken together form at least one fused ring system; and/or
R₄ and R₅ taken together form at least one fused ring system; and/or
R₅ and R taken together form at least one fused ring system; and/or
n is 1 to 30.

28. The compound according to clauses 27 wherein if R₁ is H, then R₂ is OH, and if R₁ is OH, then R₂ is H.

29. The compound according to clauses 27 or 28, wherein R₃, R⁴, R₅, and R₆ are independently H or —SO₃⁻.

30. The compound of clause 28, wherein:
R₁ is —OH; and
R₅ is SO₃⁻ or SO₃H.

31. The compound according to any of clauses 27-28, wherein R¹⁰ is —OH.

32. The compound according to clause 27, having a structure

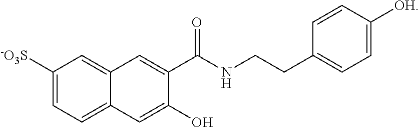

33. A compound, or a salt thereof, having a structure

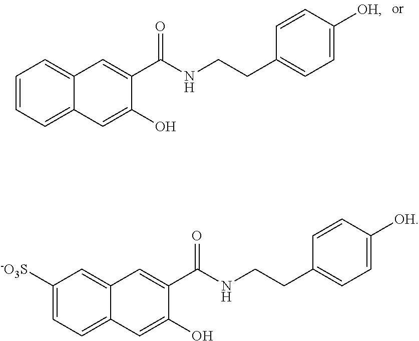

34. A compound, or a salt thereof, having a structure

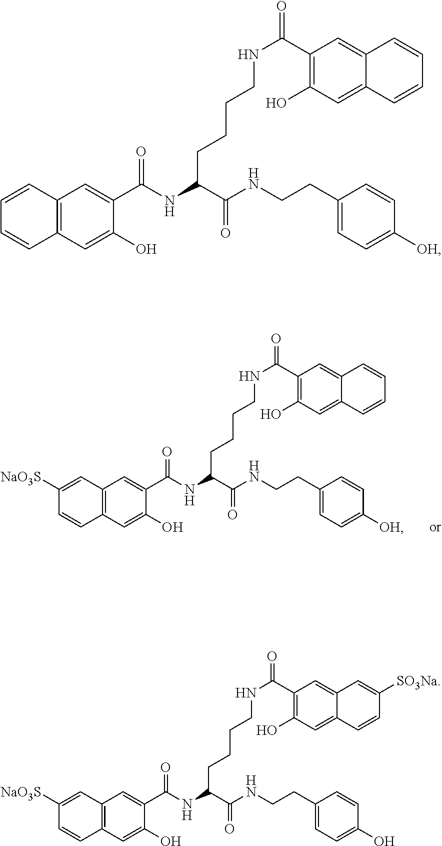

35. A covalent conjugate, comprising:
a biological sample comprising a target; and
at least one compound according to any of clauses 1-34 covalently associated with the target.

36. The conjugate according to clause 35, having a formula

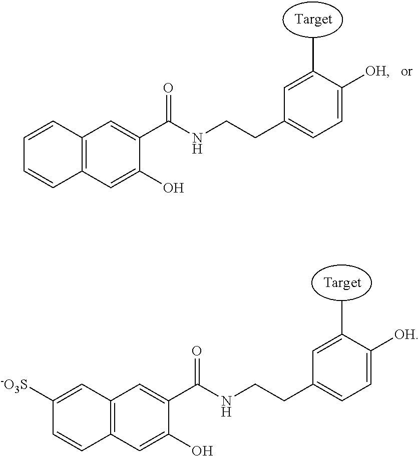

37. The conjugate according to clause 35, having a formula

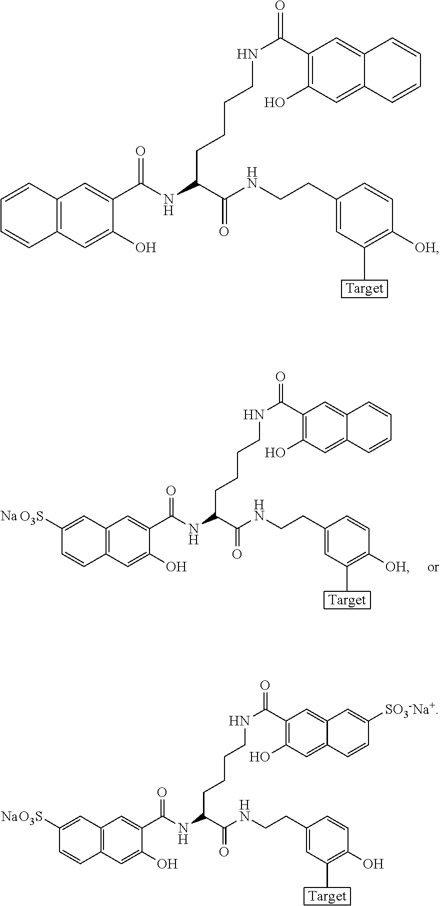

38. The conjugate according to clause 35, having a formula

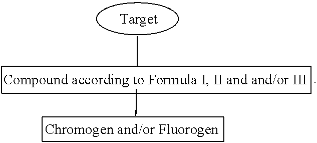

39. The conjugate according to clause 38, having a formula

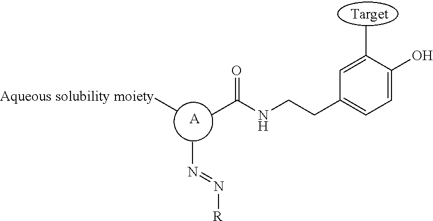

40. A method, comprising:
providing at least one compound according to any of clauses 1-34; and
using the compound to detect a target in a sample.

41. The method according to clause 40, wherein the method is an immunostaining method.

42. A method, comprising
contacting a biological sample comprising a target with a first compound according to any of clauses 1-34 under conditions selected to covalently associate the first compound to or substantially adjacent the target;
contacting the first compound with a diazonium compound under conditions that covalently link the first compound to the diazonium compound to form conjugate; and
detecting the target.

43. The method according to clause 42, wherein the first compound exhibits a first optical signal upon irradiation at an appropriate wavelength of light.

44. The method according to any of clauses 42-43, wherein the conjugate exhibits a second optical signal upon irradiation at an appropriate wavelength of light, wherein the first optical signal and the second optical signal are different.

45. The method according to any of clauses 42-44, wherein the biological target is a cell, a tissue, a virus, a gene, an epitope, a target genetic sequence, or a target protein sequence.

46. The method according to any of clauses 42-45, comprising contacting the target with the first compound in the presence of a peroxidase to covalently couple the first compound to the target.

47. The method according to clause 46, wherein the peroxidase is horseradish peroxidase or ascorbate peroxidase (APEX).

48. The method according to clause 46, wherein the peroxidase is a heme-containing peptide.

49. The method according to clause 46, wherein the peroxidase is a microperoxidase.

50. The method according to any of clauses 42-49, wherein the diazonium compound is selected from Fast Red, Fast Violet. and Fast Blue.

51. The method according to clause 42, wherein the biological target is recognized by a primary antibody, and the primary antibody is bound to a peroxidase.

52. The method according to clause 42, comprising:
contacting the biological target with a primary antibody that associates with the target; and
contacting the primary antibody with a secondary antibody to the primary antibody to form an primary-secondary antibody conjugate, wherein the secondary antibody is bound to a peroxidase.

53. The method according to any of clauses 51 or 52, wherein the primary antibody is a Ki67 antibody, an ESR1 antibody, a KRT15 antibody, and/or a CDH17 antibody.

54. The method according to clause 52, wherein:
the primary antibody is a mouse antibody, and the secondary antibody is poly HRP-goat anti-mouse (GAM); or
the primary antibody is a rabbit antibody, and the secondary antibody is poly-HRP-goat anti-rabbit (GAR).

55. A method, comprising:
contacting a biological sample having a target of interest with a primary antibody that associates with the target;
contacting the primary antibody with a secondary antibody comprising a peroxidase compound to form a primary antibody, secondary antibody conjugate;
contacting the sample with a compound satisfying Formula I

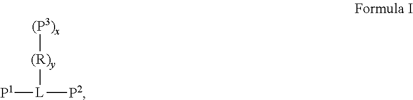

where L is a linker having from 1 to at least 50 atoms, P¹, P² and P³ are aromatic rings or aromatic ring systems. R is a bond, or is independently an L group, and x and y independently are 0 to 5, under conditions suitable for covalently bonding the compound to the sample;
contacting the sample with a diazonium salt; and
detecting the target.

56. The method according to clauses 55 wherein the target is detected colorimetrically, fluorescently, or both.

57. The method according to clauses 55 or 56, where the diazonium salt is selected from Fast Red, Fast Violet, or Fast Blue.

58. The method according to clause 55 comprising co-localizing plural different color combinations at or about the target.

59. The method according to clause 55 comprising detecting two or more targets in the sample.

60. The method according to clause 55 wherein the compound is fluorescent and the method further comprises detecting the target using fluorescence.

61. The method according to clause 55 wherein the compound has a formula P¹-L-P² and is a naphthol derivative and the diazonium salt is Fast Blue to produce a green color suitable for detecting the target.

62. The method according to clause 55 wherein the peroxidase is horseradish peroxidase or ascorbate peroxidase.

63. A kit, comprising:
a compound according to clause 1; and
a diazonium salt.

64. A kit, comprising:
a blocking buffer;
poly HRP-goat anti-mouse (GAM), poly-HRP-goat anti-rabbit (GAR), and/or HRP-conjugated streptavidin;
a compound according to clause 1;
hydrogen peroxide;
a reaction buffer;
a diazonium salt; and
a reaction stop reagent.

65. The kit according to clause 64, wherein the diazonium salt is Fast Red, Fast Blue and/or Fast Violet.

We claim:

1. A compound, satisfying Formula I 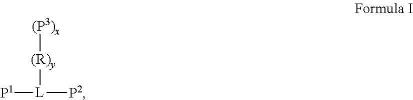
where:
L is a linker having from 1 to at least 50 atoms;
P¹, P² and P³ are aromatic rings or aromatic ring systems;
R is a bond, or is independently an L group; and
x and y independently are 0 to 5.

2. The compound according to claim 1 wherein P¹ and P³ ring systems are the same or different and each may comprise at least two fused rings, wherein at least one of the fused rings comprises a structure represented as

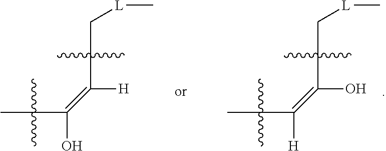

3. The compound according to claim 1 or 2, wherein P² is a phenyl ring substituted with at least one —OH group.

4. The compound according to claim 1 wherein:
L is a linker having from 2 to 20 atoms in a chain;
R is a bond, or is independently an L group; and
x and y independently are 0 or 1.

5. The compound according to claim 1, having a structure selected from

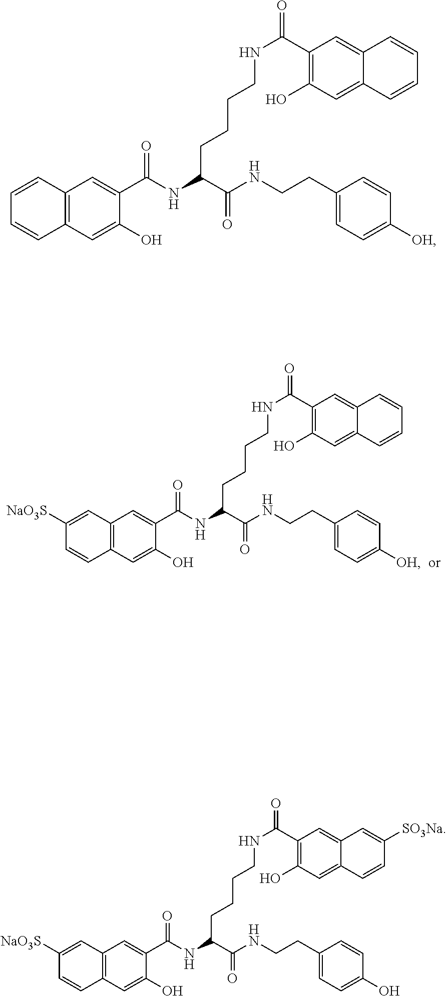

6. The compound according to claim 1, satisfying Formula II
P¹-L-P², Formula II
where:
P¹ is a first aromatic ring system comprising at least two fused rings, wherein at least one of the fused rings comprises a structure represented as 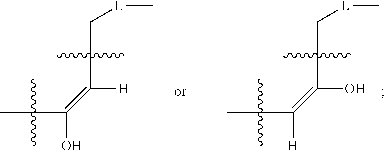
P² is a second aromatic ring system substituted with at least one —OH group; and
L is a linker.

7. The compound according to claim 6, wherein P¹ has from 2 to 4 rings in a fused-ring system.

8. The compound according to claim 1, wherein P¹ and/or P³ is substituted with at least one aqueous solubility moiety selected to increase the aqueous solubility of the compound.

9. The compound according to claim 1, wherein P¹ is selected from: 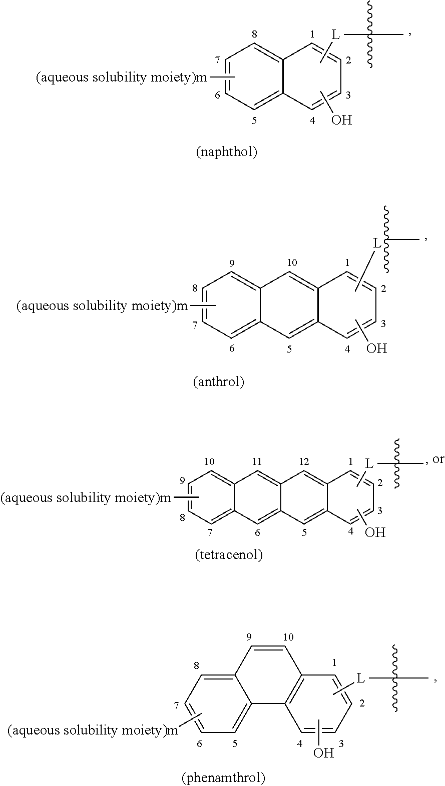
where m is from 1 to 5.

10. The compound according to claim 8 or 9, wherein the aqueous solubility moiety is selected from SO₃⁻, —SO₃H, —CO₂⁻, —CO₂H, —HPO₃⁻², H₂PO₃⁻¹, H₃PO₃, PEG,

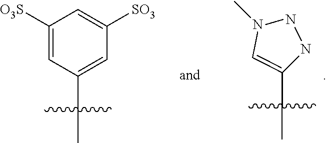

11. The compound according to claim 1 where a hydroxyl group on P¹ is ortho or meta to L.

12. The compound according to claim 9, wherein:
P¹ is naphthol, L is coupled to position 2 or 3, at least one hydroxyl group is at positions 3 and/or 4, and the at least one moiety selected to increase the aqueous solubility of the compound is coupled to at least one of the 5-8 positions;
P¹ is anthrol, L is coupled to position 2 or 3, at least one hydroxyl group is at positions 3 and/or 4, and the at least one moiety selected to increase the aqueous solubility of the compound is at coupled to at least one of the 6-9 positions;
P¹ is chrysenol, L is coupled to position 2 or 3, at least one hydroxyl group is at positions 3 and/or 4, and the at least one moiety selected to increase the aqueous solubility of the compound is at coupled to at least one of the 7-10 positions; or
P¹ is phenanthrol, L is coupled to position 2 or 3, at least one hydroxyl group is at positions 3 and/or 4, and the at least one moiety selected to increase the aqueous solubility of the compound is at coupled to at least one of the 5-8 positions.

13. The compound according to claim 1 where P¹ and/or P³ is a heteroaryl fused ring system.

14. The compound according to claim 13 where the heteroaryl fused ring system is electron rich.

15. The compound according to claim 13 or 14 wherein the heteroaryl fused ring system is selected from fused furan, fused thiophene, and fused pyrrole ring systems.

16. The compound according to claim 13, where P¹ and/or P³ is selected from

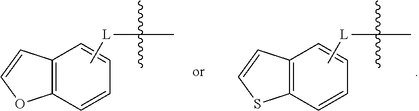

17. The compound according to claim 13, wherein P¹ and/or P³ is substituted with at least one moiety selected to increase the aqueous solubility of the compound.

18. The compound according to claim 13, wherein P² is an aromatic hydrocarbon.

19. The compound according to claim 1, wherein P² comprises a phenol.

20. The compound according to claim 19 wherein the phenol has a hydroxyl group that is para to L.

21. The compound according to claim 1, wherein L is selected from C₁-C₅₀ aliphatic, C₁-C₅₀ alkyl, C₁-C₅₀ alkyl amide, C₁-C₅₀ alkyl ester, C₁-C₅₀ ketone, a C₂-C₅₀ polyol, C₂-C₅₀ polyalkyl ethers, and C₂-C₅₀ aliphatic amines.

22. The compound according to claim 1, wherein:
P¹ is selected from 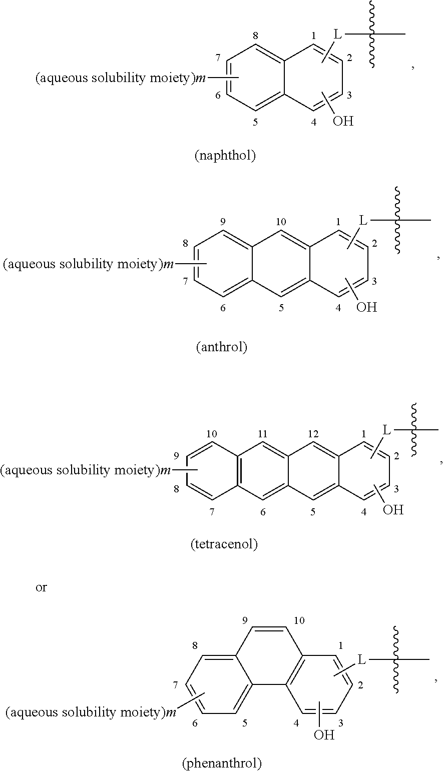
where m is from 1 to 3;
P² comprises a phenol;
the at least one moiety selected to increase the aqueous solubility of the compound is selected from —SO₃⁻, —SO₃H, —CO₂⁻, —CO₂H, —HPO₃⁻², H₂PO₃⁻¹, H₃PO₃, PEG, 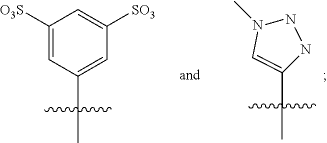
and
L is selected from a covalent bond, C₁-C₁₀ alkyl, a C₁-C₁₀ alkyl amide, a C₁-C₁₀ ketone, or a C₁-C₁₀ glycol.

23. The compound according to claim 22, where:
P¹ is naphthol;
m is 1; and
the at least one moiety selected to increase the aqueous solubility is —SO₃⁻.

24. The compound according to claim 21, where L is 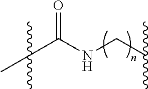
and n is from 1 to 5.

25. The compound according to claim 21, where m is 1 and n is 2.

26. The compound according to claim 1, where:
P¹ is naphthol;
P² is phenol;
the at least one moiety selected to increase the aqueous solubility is —SO₃⁻; and
L is

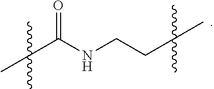 [SMILES: *CCNC(*)=O]

27. A compound, having a structure 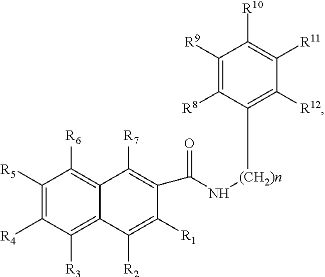
where:
R¹ and R² are independently selected from H and OH;
R³-R⁸ are independently selected from H, —F, —Cl, —Br, —I, —OH, —OR⁹ where R⁹ is C₁-C₆ alkyl, —SO₃⁻, —SO₃H, —CO₂⁻, —CO₂H, —HPO₃⁻², H₂PO₃⁻¹, H₃PO₃, PEG, 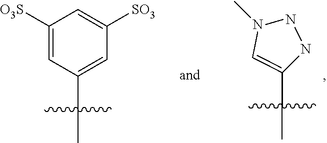
where at least one of R¹, R², R⁷ and at least one of R⁸-R¹² is hydroxyl; or
R₃ and R₄ taken together form at least one fused ring system; and/or
R₄ and R₅ taken together form at least one fused ring system; and/or
R₅ and R taken together form at least one fused ring system; and/or
n is 1 to 30.

28. The compound according to claim 27 wherein if R₁ is H, then R₂ is OH, and if R₁ is OH, then R₂ is H.

29. The compound according to claim 27 or claim 28, wherein R₃, R₄, R₅, and R₆ are independently H or —SO₃⁻.

30. The compound of claim 28, wherein:
R₁ is —OH; and
R₅ is SO₃⁻ or SO₃H.

31. The compound according to claim 27, wherein R¹⁰ is —OH.

32. The compound according to claim 27, having a structure

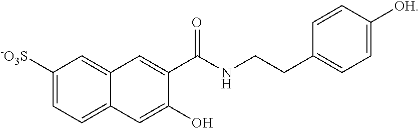

33. A compound, or a salt thereof, having a structure

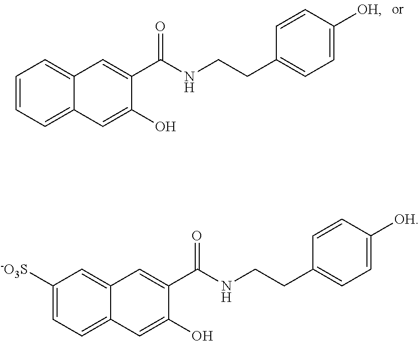

34. A compound, or a salt thereof, having a structure

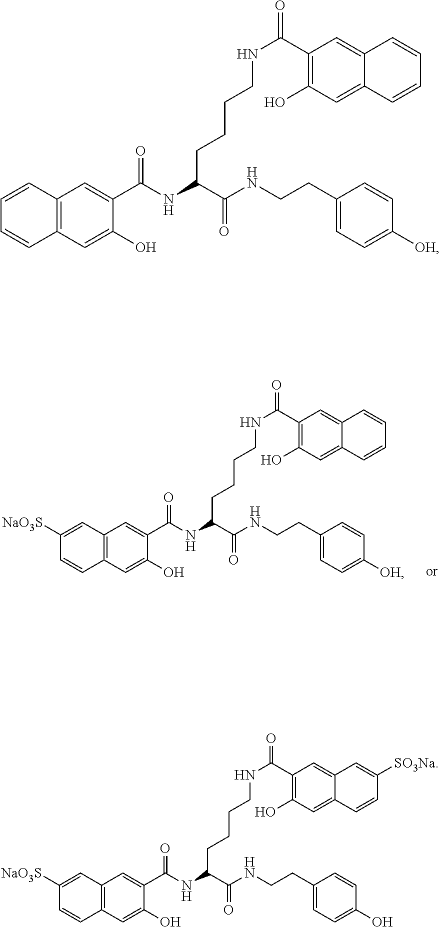

35. A covalent conjugate, comprising:
a biological sample comprising a target; and
at least one compound according to claim 1 covalently associated with the target.

36. The conjugate according to claim 35, having a formula

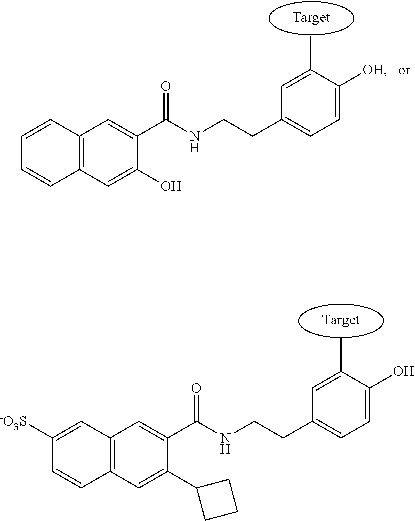

37. The conjugate according to claim 35, having a formula

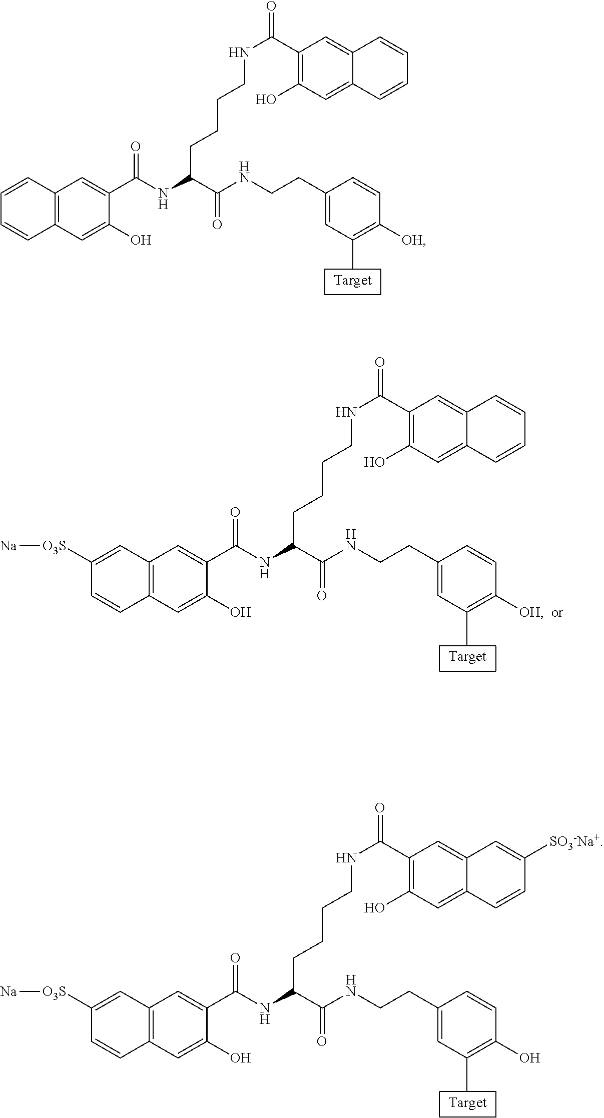

38. The conjugate according to claim 35, having a formula

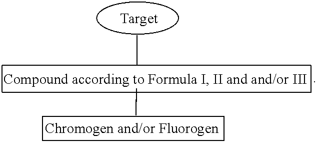

39. The conjugate according to claim 38, having a formula

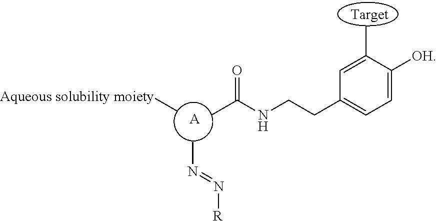

40. A method, comprising:
providing at least one compound according to claim 1; and
using the compound to detect a target in a sample.

41. The method according to claim 40, wherein the method is an immunostaining method.

42. A method, comprising
contacting a biological sample comprising a target with a first compound according to claim 1 under conditions selected to covalently associate the first compound to or substantially adjacent the target;
contacting the first compound with a diazonium compound under conditions that covalently link the first compound to the diazonium compound to form conjugate; and
detecting the target.

43. The method according to claim 42, wherein the first compound exhibits a first optical signal upon irradiation at an appropriate wavelength of light.

44. The method according to claim 42, wherein the conjugate exhibits a second optical signal upon irradiation at an appropriate wavelength of light, wherein the first optical signal and the second optical signal are different.

45. The method according to claim 42, wherein the biological target is a cell, a tissue, a virus, a gene, an epitope, a target genetic sequence, or a target protein sequence.

46. The method according to claim 42, comprising contacting the target with the first compound in the presence of a peroxidase to covalently couple the first compound to the target.

47. The method according to claim 46, wherein the peroxidase is horseradish peroxidase or ascorbate peroxidase (APEX).

48. The method according to claim 46, wherein the peroxidase is a heme-containing peptide.

49. The method according to claim 46, wherein the peroxidase is a microperoxidase.

50. The method according to claim 42, wherein the diazonium compound is selected from Fast Red, Fast Violet, and Fast Blue.

51. The method according to claim 42, wherein the biological target is recognized by a primary antibody, and the primary antibody is bound to a peroxidase.

52. The method according to claim 42, comprising:
contacting the biological target with a primary antibody that associates with the target; and
contacting the primary antibody with a secondary antibody to the primary antibody to form an primary-secondary antibody conjugate, wherein the secondary antibody is bound to a peroxidase.

53. The method according to any of claim 51 or 52, wherein the primary antibody is a Ki67 antibody, an ESR1 antibody, a KRT15 antibody, and/or a CDH17 antibody.

54. The method according to claim 52, wherein:
the primary antibody is a mouse antibody, and the secondary antibody is poly HRP-goat anti-mouse (GAM); or
the primary antibody is a rabbit antibody, and the secondary antibody is poly-HRP-goat anti-rabbit (GAR).

55. A method, comprising:
contacting a biological sample having a target of interest with a primary antibody that associates with the target;
contacting the primary antibody with a secondary antibody comprising a peroxidase compound to form a primary antibody, secondary antibody conjugate;
contacting the sample with a compound satisfying Formula I 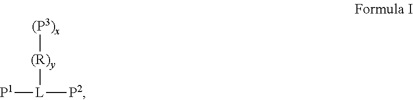
where L is a linker having from 1 to at least 50 atoms, P¹, P² and P³ are aromatic rings or aromatic ring systems, R is a bond, or is independently an L group, and x and y independently are 0 to 5, under conditions suitable for covalently bonding the compound to the sample;
contacting the sample with a diazonium salt; and
detecting the target.

56. The method according to claim 55 wherein the target is detected colorimetrically, fluorescently, or both.

57. The method according to claim 55 or 56, where the diazonium salt is selected from Fast Red, Fast Violet, or Fast Blue.

58. The method according to claim 55 comprising co-localizing plural different color combinations at or about the target.

59. The method according to claim 55 comprising detecting two or more targets in the sample.

60. The method according to claim 55 wherein the compound is fluorescent and the method further comprises detecting the target using fluorescence.

61. The method according to claim 55 wherein the compound has a formula P¹-L-P² and is a naphthol derivative and the diazonium salt is Fast Blue to produce a green color suitable for detecting the target.

62. The method according to claim 55 wherein the peroxidase is horseradish peroxidase or ascorbate peroxidase.

63. A kit, comprising:
a compound according to claim 1; and
a diazonium salt.

64. A kit, comprising;
a blocking buffer;
poly HRP-goat anti-mouse (GAM), poly-HRP-goat anti-rabbit (GAR), and/or HRP-conjugated streptavidin;
a compound according to claim 1;
hydrogen peroxide;
a reaction buffer;
a diazonium salt; and
a reaction stop reagent.

65. The kit according to claim 64, wherein the diazonium salt is Fast Red, Fast Blue and/or Fast Violet.
